(19) United States
(12) Patent Application Publication
(10) Pub. No.: US 2017/0305981 A1
(43) Pub. Date: Oct. 26, 2017

(54) SYNTHETIC PEPTIDES

(71) Applicant: The University of Chicago, Chicago, IL (US)

(72) Inventor: John Bela Ancsin, Chicago, IL (US)

(21) Appl. No.: 15/528,042

(22) PCT Filed: Nov. 20, 2015
(86) PCT No.: PCT/US15/61845

§ 371 Date: May 18, 2017

Related U.S. Application Data
(60) Provisional application No. 62/082,902, filed on Nov. 21, 2014.

(51) Int. Cl. C07K 14/47; A61K 38/17; C12N 9/20; A61K 38/00

(52) U.S. Cl. CPC C07K 14/47; C12N 9/20; A61K 38/1709; A61K 38/00

(57) ABSTRACT

Provided herein are compositions comprising synthetic lipase-stimulating peptides, and methods of treating hyper-triglyceridemia and other conditions and diseases therewith. In particular, synthetic peptides (AV-peptides) and peptidomimetics (AV-peptidomimetics) are provided that exhibit the lipase-stimulating activity of apoA-V or an enhancement thereof, as well as methods of use thereof. Provided herein are compositions comprising a peptide or polypeptide having less than 100% sequence identity with full length ApoA-V, encompassing a portion with at least 50% sequence identity with AV199-224.

The present application claims priority to U.S. Provisional application Ser. No. 62/082,902, filed Nov. 21, 2014, which is herein incorporated by reference in its entirety.

### FIELD

Provided herein are compositions comprising synthetic lipase-stimulating peptides, and methods of treating hypertriglyceridemia and other conditions and diseases therewith. In particular, synthetic peptides (AV-peptides) and peptidomimetics (AV-peptidomimetics) are provided that exhibit the lipase-stimulating activity of apoA-V or an enhancement thereof, as well as methods of use thereof.

### BACKGROUND

Over 350 million people across the major pharmaceutical markets are affected by hypertriglyceridemia (HTG) (TG levels of >150 mg/dL; 100 mg/dL is normal), which is an important risk factor for several diseases including cardiovascular disease, pancreatitis, diabetes, and hepatic steatosis. About 1-2% of the population have genetically inherited primary HTG (>500 mg/dL TG), which can be either a monogenic or polygenic disorder associated with serious clinical morbidity and mortality outcomes. Current treatments for HTG have numerous off-target effects and do not sufficiently meet patient's recommended goals.

### SUMMARY

Provided herein are compositions comprising synthetic lipase-stimulating peptides, and methods of treating hypertriglyceridemia and other conditions and diseases therewith. In particular, synthetic peptides (AV-peptides) and peptidomimetics (AV-peptidomimetics) are provided that exhibit the lipase-stimulating activity of apoA-V or an enhancement thereof, as well as methods of use thereof.

Provided herein are compositions comprising a peptide or polypeptide having less than 100% sequence identity with SEQ ID NO: 1 (full length ApoA-V), encompassing a portion with at least 50% sequence identity (e.g., >50%, >60%, >70%, >75%, >80%, >85%, >90%, >95%) with SEQ ID NO: 8 (AV199-224), and exhibiting lipase-stimulating activity. In some embodiments, the peptide or polypeptide comprises a portion with at least 80% sequence similarity (e.g., >80%, >90%, >95%) with SEQ ID NO: 8. In some embodiments, the peptide or polypeptide has less than 100% sequence identity, but more than 50% sequence identity (e.g., >50%, >60%, >70%, >75%, >80%, >85%, >90%, >95%) with SEQ ID NO: 3 (AV199-237). In some embodiments, the peptide or polypeptide has at least 80% sequence similarity with SEQ ID NO: 3. In some embodiments, the peptide or polypeptide has less than 100% sequence identity, but more than 50% sequence identity (e.g., >50%, >60%, >70%, >75%, >80%, >85%, >90%, >95%) with SEQ ID NO: 5 (AV199-232). In some embodiments, the peptide or polypeptide has less than 100% sequence identity with a natural apoA-V sequence (e.g., SEQ ID NO: 1 or a portion thereof (e.g., SEQ ID NO: 5)), but 50% or greater sequence identity (e.g., >50%, >60%, >70%, >75%, >80%, >85%, >90%, >95%) with SEQ ID NO: 5 (AV199-232), SEQ ID NO: 12 (AV-H/K), SEQ ID NO: 23 (AV-H/K-2F), SEQ ID NO: 26 (AV-H/K-C6S), SEQ ID NO: 27 (AV-H/K-Ac/NH₂), and/or SEQ ID NO: 28 (AV-H/K-2F/HAr).

In some embodiments, the composition comprises a peptide with less than 100% but more than 50% sequence identity (e.g., <100%, but >50%, >60%, >70%, >75%, >80%, >85%, >90%, >95%) with SEQ ID NO: 5 (AV199-232). In some embodiments, the peptide has at least 50% sequence similarity (e.g., >50%, >55%, >60%, >65%, >70%, >75% >80%, >85%, >90%, >95%) with SEQ ID NO: 5. In some embodiments, the peptide has less than 100% but more than 50% sequence identity (e.g., <100%, but >50%, >60%, >70%, >75%, >80%, >85%, >90%, >95%) with SEQ ID NO: 10. In some embodiments, the peptide has at least 80% sequence similarity (e.g., >80%, >85%, >90%, >95%, 100%) with SEQ ID NO: 10. In some embodiments, a peptide or polypeptide has a portion with 100% sequence identity with SEQ ID NO: 10. In some embodiments, a peptide is SEQ ID NO: 10. In some embodiments, the peptide has less than 100% sequence identity, but more than 50% sequence identity (e.g., <100%, but >50%, >60%, >70%, >75%, >80%, >85%, >90%, >95%) with SEQ ID NO: 11. In some embodiments, the peptide has at least 80% sequence similarity (e.g., >80%, >85%, >90%, >95%, 100%) with SEQ ID NO: 11. In some embodiments, a peptide or polypeptide has a portion with 100% sequence identity with SEQ ID NO: 11. In some embodiments, a peptide is SEQ ID NO: 11. In some embodiments, the peptide has less than 100% sequence identity, but more than 50% sequence identity (e.g., <100%, but >50%, >60%, >70%, >75%, >80%, >85%, >90%, >95%) with SEQ ID NO: 12. In some embodiments, the peptide has at least 80% sequence similarity (e.g., >80%, >85%, >90%, >95%, 100%) with SEQ ID NO: 12. In some embodiments, a peptide or polypeptide has a portion with 100% sequence identity with SEQ ID NO: 12. In some embodiments, a peptide is SEQ ID NO: 12.

In some embodiments, a synthetic AV-peptide is 10-50 amino acids in length (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, and any ranges therein (e.g., 20-48, 22-46, 24-44, 26-42, 28-40, 30-38, 32-36)). In some embodiments, a synthetic AV-peptide comprises at least 1 mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or more, and any ranges therein) from the wild-type or a natural ApoA-V sequence over the length of the peptide. In some embodiments, a synthetic AV-peptide comprises at least 1 non-conservative mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or more, and any ranges therein) from the wild-type or a natural ApoA-V sequence over the length of the peptide. In some embodiments, an AV-peptide comprises at least 1 conservative mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or more, and any ranges therein) from the wild-type or a natural ApoA-V sequence over the length of the peptide. In some embodiments, an AV-peptide comprises at least 1 semi-conservative mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or more, and any ranges therein) from the wild-type or a natural ApoA-V sequence over the length of the peptide. In some embodiments, a peptide or polypeptide is provided that comprises a synthetic AV-peptide sequence.

In some embodiments, peptides have less than 100% but greater than 50% (e.g., 55%, 60%, 70%, 80%, 90%, 95%, and any ranges therein) sequence identity to a portion of ApoA-V (e.g., SEQ ID NO: 5) that is at least 5 amino acids in length (e.g., 5, 10, 15, 20, 25, 30, 35, 40, 45, 50, and any ranges therein). In some embodiments, peptides have less than 100% but greater than 50% (e.g., 55%, 60%, 70%, 80%, 90%, 95%, and any ranges therein) sequence identity to a portion of ApoA-V (e.g., SEQ ID NO: 5) that is at least 10, 12, 14, 16, 18, 20, 22, 24, 26, 28, 30, 32, or 34 amino acids in length,

In some embodiments, compositions are provided comprising peptides and/or polypeptides that exhibit enhanced lipase-stimulating activity relative to SEQ ID NO: 1 (full length ApoA-V), for example, in one or more of the assays set forth respectively in Example 1, the description of FIGS. 3A-C, the description of FIGS. 4A-D, and the description of FIG. 8. In some embodiments, peptides and/or polypeptides exhibit >10% increased, >20% increased, >30% increased, >40% increased, >50% increased, >60% increased, >70% increased, >80% increased, >90% increased, >2-fold, >3-fold, >4-fold, >5-fold, >6-fold, >8 fold, >10-fold, or >20-fold lipase-stimulating activity relative to SEQ ID NO: 1. In some embodiments, compositions are provided comprising peptides and/or polypeptides that exhibit enhanced lipase-stimulating activity relative to SEQ ID NO: 5 (AV199-232). In some embodiments, peptides and/or polypeptides exhibit >10% increased, >20% increased, >30% increased, >40% increased, >50% increased, >60% increased, >70% increased, >80% increased, >90% increased, >2-fold, >3-fold, >4-fold, >5-fold, >6-fold, >8 fold, >10-fold, or >20-fold lipase-stimulating activity relative to one or both of SEQ ID NO: 5, for example, in one or more of the assays set forth respectively in Example 1, the description of FIGS. 3A-C, the description of FIGS. 4A-D, and the description of FIG. In some embodiments, peptides exhibit alpha-helical character. In some embodiments, secondary structure prediction techniques identify said peptides as substantially or entirely alpha helical.

In some embodiments, peptides and/or polypeptides herein comprise an amphipathic α-helix. In some embodiments, an amphipathic α-helix herein exhibits the domain organization of the AV-199-232 peptide (See, e.g., FIG. 11). The 3D structure modeling, bioinformatics, and biochemical and biophysical experiments conducted during development of embodiments herein indicate that the AV199-232 peptide and active variants thereof (and polypeptides comprising such peptides) comprise four domains, defined by amino acid classification and circumferential location on the alpha helix (e.g., spatial location in a helical wheel representation of the alpha helix), not by primary sequence. The domains of AV-199-232 and related peptides and polypeptides are: Domain 1: A1, R5, R12, L16, K19, 23A, 30D, and 34E; Domain 2: R2, C6, K13, K17, A20, R24, and Q31; Domain H: S4, T15, H22, and R33; and Domain L: L3, V7, L10, S11, L14, A18, L21, 125, N28, L29, and L32. Experiments conducted during development of embodiments herein, as well as the identity of the residues within the domains, are consistent with Domain 1 and H (and also Domain 2) having roles in stimulating Lipase activity, Domain L having a role in lipid binding or interaction, and Domain 2 having a role in heparin binding; however, embodiments herein are not limited to any particular mechanism of action and an understanding of the mechanism of action is not necessary to practice such embodiments. In some embodiments, provided herein are compositions comprising a synthetic peptide or polypeptide comprising a substantially alpha helical region having an amino acid sequence in which at least 24 amino acids (e.g., 24, 25, 26, 27, 28, 29, 30, 31, 32, and any ranges there between) and no more than 33 amino acids are identical to, or conservative or semi-conservative substitutions with, SEQ ID NO: 5. In some embodiments, the peptide or polypeptide is capable of binding to lipoprotein lipase (LpL) and/or stimulating the lipase activity of LpL. In some embodiments, at least 24 amino acids (e.g., 24, 25, 26, 27, 28, 29, 30, 31, 32, and any ranges there between) are identical to, or conservative substitutions with, SEQ ID NO: 5. In some embodiments, at least 24 amino acids (e.g., 24, 25, 26, 27, 28, 29, 30, 31, 32, and any ranges there between) are identical to SEQ ID NO: 5. In some embodiments, the peptide or polypeptide is capable of binding to and/or stimulates LpL in vitro. In some embodiments, the peptide or polypeptide is capable of binding to and/or stimulates LpL in vivo. In some embodiments, the peptide or polypeptide is capable of binding heparin.

In some embodiments, amino acids in the peptide or polypeptide corresponding to a position in SEQ ID NO: 5 are: (i) identical to the corresponding position in SEQ ID NO: 5, (ii) a conservative substitution relative to the corresponding position in SEQ ID NO: 5, (iii) a semi-conservative substitution relative to the corresponding position in SEQ ID NO: 5, and/or (iv) a non-conservative substitution relative to the corresponding position in SEQ ID NO: 5. In some embodiments, amino acids in the peptide or polypeptide corresponding to a position in SEQ ID NO: 5 are: (i) identical to the corresponding position in SEQ ID NO: 5, (ii) a conservative substitution relative to the corresponding position in SEQ ID NO: 5, and/or (iii) a semi-conservative substitution relative to the corresponding position in SEQ ID NO: 5. In some embodiments, amino acids in the peptide or polypeptide corresponding to a position in SEQ ID NO: 5 are: (i) identical to the corresponding position in SEQ ID NO: 5, and/or (ii) a conservative substitution relative to the corresponding position in SEQ ID NO: 5. In some embodiments, amino acids in the peptide or polypeptide corresponding to a position in SEQ ID NO: 5 are: (i) identical to the corresponding position in SEQ ID NO: 5. In some embodiments, any amino acids in the peptide or polypeptide corresponding a position in SEQ ID NO: 5 that are not identical to the corresponding position in SEQ ID NO: 5, and are not otherwise specified as being a particular amino acid or selected from a set of amino acids, are conservative substitutions or semi-conservative substitutions relative to the corresponding positions in SEQ ID NO: 5. In some embodiments, any amino acids in the peptide or polypeptide corresponding a position in SEQ ID NO: 5 that are not identical to the corresponding position in SEQ ID NO: 5, and are not otherwise specified as being a particular amino acid or selected from a set of amino acids, are conservative substitutions relative to the corresponding positions in SEQ ID NO: 5.

In some embodiments, provided herein are compositions comprising a synthetic peptide or polypeptide described herein comprising a domain of grouped residues (Domain 2) and comprising residues corresponding to positions 2, 6, 13, 17, 20, 24, and 31 of SEQ ID NO: 5. In some embodiments, Domain 2 is capable of binding to heparin. In some embodiments, the residue in the peptide or polypeptide corresponding to position 6 of SEQ ID NO: 5 is not a cysteine residue. In some embodiments, the residue corresponding to position 6 of SEQ ID NO: 5 is the only residue in Domain 2 that is not identical to corresponding positions in SEQ ID NO: 5. In some embodiments, the residue in the peptide or polypeptide corresponding to position 6 of SEQ ID NO: 5 is selected from the group consisting of S, T, N, and Q. In some embodiments, the residue in the peptide or polypeptide corresponding to position 6 of SEQ ID NO: 5 is S. In some embodiments, the residue in the peptide or polypeptide corresponding to position 6 of SEQ ID NO: 5 is T. In some embodiments, the residue in the peptide or polypeptide corresponding to position 6 of SEQ ID NO: 5 is selected from the group consisting of Y and H. In some embodiments, one or both residues in the peptide or polypeptide corresponding to positions 13 and 17 of SEQ ID NO: 5 are selected from the group consisting of homolysine (hLys), R, homoarginine (hArg), and ornithine. In some embodiments, the residue in the peptide or polypeptide corresponding to position 17 of SEQ ID NO: 5 is selected from the group consisting of Q, N, and S.

In some embodiments, provided herein are compositions comprising a synthetic peptide or polypeptide described herein comprising a domain of grouped residues (Domain H) comprising residues corresponding to positions 4, 15, 22, and 33 of SEQ ID NO: 5. In some embodiments, the residue in the peptide or polypeptide corresponding to position 22 of SEQ ID NO: 5 is not a histidine residue. In some embodiments, the residue in the peptide or polypeptide corresponding to position 22 of SEQ ID NO: 5 is selected from the group consisting of K, hLys, R, hArg, and ornithine. In some embodiments, the residue corresponding to position 22 of SEQ ID NO: 5 is K. In some embodiments, the residue corresponding to position 22 of SEQ ID NO: 5 is hLys. In some embodiments, the residue in the peptide or polypeptide corresponding to position 4 of SEQ ID NO: 5 is S. In some embodiments, the residue in the peptide or polypeptide corresponding to position 4 of SEQ ID NO: 5 is selected from the group consisting of T, N, or G. In some embodiments, the residues in the peptide or polypeptide corresponding to positions 15 and 33 of SEQ ID NO: 5 are T and R, respectively.

In some embodiments, provided herein are compositions comprising a synthetic peptide or polypeptide described herein comprising a hydrophobic domain of grouped residues (Domain L) comprising residues corresponding to positions 3, 7, 10, 11, 14, 18, 21, 25, 28, 29, and 32 of SEQ ID NO: 5. In some embodiments, one or more residues of the hydrophobic domain are non-identical to corresponding to positions of SEQ ID NO: 5. In some embodiments, one or more residues of the hydrophobic domain corresponding to non-polar aliphatic residues of SEQ ID NO: 5 are substituted with nonpolar aromatic residues selected from the group consisting of F, Y, and W. In some embodiments, the residue in the peptide or polypeptide corresponding to position 10 of SEQ ID NO: 5 is a nonpolar aromatic residue. In some embodiments, the residue in the peptide or polypeptide corresponding to position 10 of SEQ ID NO: 5 is F. In some embodiments, the residue in the peptide or polypeptide corresponding to position 29 of SEQ ID NO: 5 is a nonpolar aromatic residue. In some embodiments, the residue in the peptide or polypeptide corresponding to position 29 of SEQ ID NO: 5 is F. In some embodiments, the peptide or polypeptide comprises one or more residues in the hydrophobic domain corresponding to positions in SEQ ID NO: 5 that are non-identical to the residues at the corresponding positions in SEQ ID NO: 5. In some embodiments, at least one of the non-identical positions in the hydrophobic domain is a Y. In some embodiments, at least two of the non-identical positions in the hydrophobic domain are Y. In some embodiments, at least two of the non-identical positions in the hydrophobic domain are selected from the group consisting of F, Y, and W. In some embodiments, at least one of the non-identical positions in the hydrophobic domain is F. In some embodiments, at least two of the non-identical positions in the hydrophobic domain are F. In some embodiments, two of the non-identical positions in the hydrophobic domain are L10F and L29F. In some embodiments, at least one of the non-identical positions in the hydrophobic domain is a W. In some embodiments, at least two of the non-identical positions in the hydrophobic domain are W. In some embodiments, at least three of the non-identical amino acids in the hydrophobic domain are substitutions of a non-polar aliphatic amino acid with a nonpolar aromatic amino acid selected from the group consisting of F, Y, and W. In some embodiments, at least one of the non-identical amino acids in the hydrophobic domain is a substitution of a non-polar aliphatic amino acid with a different non-polar aliphatic amino acid.

In some embodiments, provided herein are compositions comprising a synthetic peptide or polypeptide described herein comprising a domain of grouped residues (Domain 1) comprising residues corresponding to positions 1, 5, 12, 16, 19, 23, 30, and 34 of SEQ ID NO: 5. In some embodiments, one or more residues within Domain 1 corresponding to lysine residues of SEQ ID NO: 5 are substituted with a residue selected from the group consisting of R, hArg, and hLys. In some embodiments, one or more residues within Domain 1 corresponding to arginine residues of SEQ ID NO: 5 are substituted with a residue selected from the group consisting of K, hArg, and hLys. In some embodiments, all residues in Domain 1 are identical to corresponding residues in SEQ ID NO: 5 other than one or more arginine or lysine residues of SEQ ID NO: 5 which are substituted with a residue selected from the group consisting of R, K, hArg, and hLys. In some embodiments, at least one of the non-identical amino acids is a substitution of an arginine with hArg or hLys.

In some embodiments, provided herein are peptides and polypeptides comprising a sequence having 10 or fewer (e.g., 10, 9, 8, 7, 6, 5, 4, 3, 2, 1, 0, or ranges there between) substitutions (e.g., conservative substitutions, semi-conservative substitutions, non-conserved substitutions) relative to the sequence:
AR(L/F/Y/W)(S/T/N/G)(R/K/hLys/hArg)(C/S/T/N/Q/Y/H)(V/F/Y/W)QV(L/F/Y/W)S(R/K/h Lys/hArg)(K/R/hArg/hLys/Orn)(L/F/Y/W)TL(K/R/hArg/hLys/Orn)(A/F/Y/W)(R/K/hLys/hA rg)A(L/F/Y/W)(H/K/hLys/R/hArg/Orn)AR(I/F/Y/W)QQN(L/F/Y/W)DQ(L/F/Y/W)RE (SEQ ID NO: 29), but less than 100% sequence identity with SEQ ID NO: 5. In some embodiments, provided herein are peptides and polypeptides comprising a sequence having 10 or fewer (e.g., 10, 9, 8, 7, 6, 5, 4, 3, 2, 1, 0, or ranges there between) substitutions (e.g., conservative substitutions, semi-conservative substitutions, non-conserved substitutions) relative to the sequence:
ARX₃X₄X₅X₆X₇QVX₁₀SX₁₂X₁₃X₁₄TLX₁₇X₁₈X₁₉AX₂₁X₂₂ARX₂₅QQNX₂₉DQX₃₂RE, wherein: X₃ is L, F, Y, or W; X₄ is S, T, N, or G; X₅ is R, K, hLys, or hArg; X₆ is C, S, T, N, Q, Y, or H; X₇ is V, F, Y, or W; X₁₀ is L, F, Y, or W; X₁₂ is R, K, hLys, or hArg; X₁₃ is K, R, hArg, hLys, or Orn; X₁₄ is L, F, Y, or W; X₁₇ is K, R, hArg, hLys, or Orn; X₁₈ is A, F, Y, or W; X₁₉ is R, K, hLys, or hArg; X₂₁ is L, F, Y, or W; X₂₂ is H, K, hLys, R, hArg, or Orn; X₂₅ is I, F, Y, or W; X₂₉ is L, F, Y, or W; and X₃₂ is L, F, Y, or W (SEQ ID NO: 29), but less than 100% sequence identity with SEQ ID NO: 5. In some embodiments, provided herein are peptides and polypeptides comprising a sequence having 100% sequence identity with SEQ ID NO: 29, but less than 100% sequence identity with SEQ ID NO: 5.

In some embodiments, provided herein are compositions comprising a synthetic peptide or polypeptide described herein comprising an N-terminal modification selected from the group consisting of: desamino, N-lower alkyl, N-di-lower alkyl, constrained alkyl and N-acyl modifications. In some embodiments, the C-terminus of the peptide or polypeptide comprises a modification selected from the group consisting of: amide, lower alkyl amide, constrained alkyls, dialkyl amide, and lower alkyl ester modification.

In some embodiments, provided herein are pharmaceutical preparations comprising: (a) an AV-peptide or polypeptide described herein (e.g., in the preceding paragraphs); and (b) a physiologically acceptable buffer or carrier. In some embodiments, pharmaceutical preparations further comprise an additional therapeutic agent (e.g., for the treatment of HTG, arthrosclerosis, elevated cholesterol, heart disease, etc.).

In some embodiments, provided herein are fusion peptides or polypeptides comprising: (a) an AV-peptide or polypeptide described herein (e.g., in the preceding paragraphs), and (b) a functional peptide or polypeptide segment. In some embodiments, the functional peptide or polypeptide segment comprises a signaling moiety, therapeutic moiety, localization moiety (e.g., cellular import signal, nuclear localization signal, etc.), detectable moiety (e.g., fluorescent moiety, contrast agent), or isolation/purification moiety (e.g., streptavidin, His₆, etc.).

In some embodiments, provided herein are polynucleotides encoding an AV-peptide or polypeptide described herein (e.g., in the preceding paragraphs). In some embodiments, provided herein are nucleic acid vectors (e.g., plasmid, bacmid, viral vector (e.g., AAV) comprising polynucleotides encoding an AV-peptide or polypeptide described herein (e.g., in the preceding paragraphs). In some embodiments, vectors further comprise a promoter and/or one or more expression elements (e.g., transcription enhancer, translational start site, internal ribosome entry site, etc.). In some embodiments, methods are provided comprising administering a polynucleotide or vector described herein to a subject or sample (e.g., for the treatment of hypertriglyceridemia, for reducing concentration of triglycerides, etc.).

In some embodiments, provided herein are methods of treating hypertriglyceridemia or a related condition or disease (e.g., atherosclerosis, heart disease, acute pancreatitis, etc.) comprising administering an AV-peptide or polypeptide described herein (e.g., in the preceding paragraphs) to a subject suffering from hypertriglyceridemia or said related condition or disease.

In some embodiments, provided herein are methods of preventing hypertriglyceridemia or a related condition or disease (e.g., atherosclerosis, heart disease, acute pancreatitis, etc.) comprising administering an AV-peptide or polypeptide described herein (e.g., in the preceding paragraphs) to a subject at risk (e.g., family history, genetic predisposition, elevated triglycerides, lifestyle, age, gender, etc.) of hypertriglyceridemia or said related condition or disease.

In some embodiments, provided herein are methods of treating or preventing atherosclerosis and/or cardiovascular disease in a subject comprising administering an AV-peptide or polypeptide described herein (e.g., in the preceding paragraphs) to a subject. In some embodiments, administering comprises co-administering: (a) said peptide or polypeptide, and (b) a therapy and/or therapeutic for the treatment and/or prevention of atherosclerosis and/or cardiovascular disease. In some embodiments, (a) and (b) are administered simultaneously and/or in a single pharmaceutical preparation. In some embodiments, (a) and (b) are administered concurrently and/or in separate pharmaceutical preparations.

In some embodiments, provided herein are methods of reducing triglyceride concentration in a sample comprising: (a) administering an AV-peptide or polypeptide described herein (e.g., in the preceding paragraphs) to a sample comprising (i) triglycerides, and (ii) triglyceride-hydrolyzing lipases; or (b) administering: (i) an AV-peptide or polypeptide described herein (e.g., in the preceding paragraphs), and (ii) triglyceride-hydrolyzing lipases to a sample comprising triglycerides. In some embodiments, the triglyceride-hydrolyzing lipases are selected from the group consisting of lipoprotein lipase (LpL), endothelial lipase (EL) and hepatic lipase (HL).

In some embodiments, provided herein is the use of the AV-peptide or polypeptides described herein for the treatment of hypertriglyceridemia or a related condition or disease (e.g., atherosclerosis, heart disease, acute pancreatitis, etc.). In some embodiments, provided herein are the AV-peptide or polypeptides described herein for use as a medicament. In some embodiments, provided herein are AV-peptide or polypeptides for use in the treatment of hypertriglyceridemia or a related condition or disease (e.g., atherosclerosis, heart disease, acute pancreatitis, etc.). In some embodiments, provided herein is the use of the AV-peptide or polypeptides described herein for the manufacture of a medicament for the treatment of hypertriglyceridemia or a related condition or disease (e.g., atherosclerosis, heart disease, acute pancreatitis, etc.).

### BRIEF DESCRIPTION OF THE DRAWINGS

FIGS. 1A-D. (A) Human apoA-V protein sequence (mature protein; uniprot/Q6Q788; SEQ ID NO: 1) highlighting a deletion and several mutations that have been identified in various populations and associated with hypertriglyceridemia (HTG); (B) ApoA-V loss of function mutations/deletions cluster within residues 199-259 indicating that this region may harbor a potential LpL stimulating domain. Mapping all known sequence variants of apoA-V linked to hypertriglyceridemia and/or CAD cluster within residues 199-259, mutations vertical lines and asterisks represent mutations previously reported to reduce LpL stimulating activity of apoA-V; (C) a 3D model for apoA-V (Phyre2) predicts a structure rich in α-helix; and (D) test peptide AV199-237 contains a putative leucine-zipper motif (SEQ ID NO: 20).

FIG. 2A-B. Predicted 3-D structure of apoA-V by Phyre2 highlighting location of the (Ser232_Leu235)del and leu253Pro SNPs (A). Protein sequence shown (SEQ ID NO: 2) (B) bracketed by sequences rich in P and A, which are known protein structure disruptors (α-helix breaker). Template for the synthetic peptide AV199-237 (SEQ ID NO: 3) was chosen by visual inspection. Peptide numbering is based on the mature protein (signal peptide removed).

FIG. 3A-C. Lipoprotein lipase (LpL) activity assay using a fluorogenic TG analog, EnzChek (Invitrogen). The enzyme reaction mix was composed of increasing concentrations of LpL in 100 μl Tris buffered saline (TBS), 1% BSA, pH 7.8, 0.015% Zwittergent 3-14 and 1 μM EnzChek substrate. Reaction was monitored by fluorescence (λex=482 nm, λem=515 nm) and the relative fluorescence units (RFU) against time plotted (A). BODIPY labeled fatty acid (BODIPY-FA) was used to determine product concentration generated with the lipase (B). Structure of the EnzChek substrate (E33955) which was purchased from Invitrogen Inc. (C).

FIG. 4. Lipase activity assayed in hepatocyte (HepG2) and monocyte (RAW267.4) cell lines. In HepG2 cultures, reaction was dependent on cell surface contact of the TG-substrate. Removal of the reaction media from the cells halted further production of product and returning it to the cell re-initiated the reaction (A). Pre-wash of the HepG2 cells with heparin, which is known to remove the lipases from the cell surface, also reduced the rate of hydrolysis of the TG-substrate (B). Both cells (C) and pure LpL (D) were sensitive to the apo-protein content of different lipoproteins (VLDL, LDL and HDL).

FIG. 5. The AV199-237 peptide stimulated LpL activity but only in the presence of TG-rich lipoproteins. Peptide (10 μM) added to LpL did not increase enzyme activity (panel A), unless the peptide was first incubated with either VLDL or LDL (B). The addition of either VLDL or LDL caused a reduction in LpL activity due to the apoC-III content. AV199-237 LpL-stimulating activity was compared the apoC-II peptide (C-II50-79), another activator of LpL (C). While the AV199-237 peptide stimulated LpL activity in the presence of VLDL, AV250-288 did not (D,E).

FIG. 6. The AV199-237 peptide was effective in stimulating the activity of hepatic lipase (HL) in live hepatocyte cells in culture (A). The addition of AV199-237 (10 μM) to the cells increased HL activity by approximately 2.8-fold and was sustained for the full 75 min period the reaction was monitored (B).

FIG. 7. The AV residues 199-288 were screened for lipase stimulating activity to determine the minimum peptide sequence necessary to stimulate LpL activity. Relative activities are indicated with ++, +, and −.

FIG. 8. Determining the minimum peptide sequence (A) harboring full lipase stimulating activity (B, D). Shortening AV199-237 by 5 residues to AV199-232 produced enhancement of activity while reducing the length further had a negative effect on activity. Assays were performed by adding 10 μM of peptide to in vitro LpL assays (B) or to hepatocyte (HepG2) cell cultures (D). In 24 well tissue culture plates, cells were incubated for 1 hr at 37° C. in 200 μL TBS, 1.5% BSA, 0.2% zwittergent 3-14, 1μM EnzChek substrate. AV199-232 was also active in cultures of live THP-1 macrophages known to express LpL (C).

FIG. 9. AV199-232 stimulates LpL activity by improving its affinity for substrate. Lipase reaction was performed with different substrate concentrations (0-25 μM) (A, C, D) to determine the kinetic parameters of LpL enzymatic activity, +/− VLDL (10 μg/ml) and +/− AV199-232 (2.5 μM). (B) The addition of VLDL reduced the enzymes affinity for substrate by 5.3-fold which is consistent with inhibition by apoC-III. AV199-232 showed only marginal changes in the Vₘₐₓ and Kₘ. AV199-232+VLDL increases LpL's apparent affinity for substrate by 7.8-fold. Kinetic parameters were determined using GraphPad Prism software curve fitting to the Michaelis-Menten enzyme kinetic equation.

FIG. 10. AV199-232 is an a-helical peptide that binds heparin. Peptide folding and conformation was analyzed experimentally by circular dichroism spectroscopy (A) and calculated to be ˜42% α-helical which increased to ˜76% on binding heparin. 3D-modeling in silico using Phyre2 also indicated that the peptide energetically can fold into an α-helix (B).

FIG. 11. Structural representations of AV199-232: (A) 3D structure model, predicted to be >88% α-helical (predictprotein.org), in agreement with circular dichroism analysis; (B) Helical wheel representation generated using Heliquest2 freeware reveals four distinct domains (1, 2, H, and L); and (C) primary sequence of AV199-232, highlighting some key residues of interest in each of the individual domains.

FIG. 12. To investigate the importance of the domains peptides with select residue substitutions were synthesized to “knockout” the domains individually.

FIG. 13. Lipase stimulating (A) and heparin binding (B) potentials for the AV199-232 peptides with knockout substitutions in domain 1 and domain 2. Lipase activity appears highly dependent on both domain 1 and domain 2, while heparin binding requires domain 2.

FIG. 14. The substitution of H residue in domain-H with K (AV-H/K) substantially improved the lipase stimulating potential of AV199-232. In the absence of VLDL, AV-H/K stimulated LpL activity by 1.6-fold (A/C). When incubated with VLDL before added to LpL, AV-H/K stimulated LpL activity by 8-fold, a 44% increase over AV199-232 (B/D). All peptides were compared at 10 μM concentration.

FIG. 15. AV199-232 binds VLDL (A) and the peptide-lipoprotein complex stimulates LpL activity (B).

FIG. 16. Tuning the lipid-binding face improves lipase stimulating performance.

FIG. 17. Summary of exemplary AV-peptides tested for lipase stimulating activity. Sequences are shown with relative influences on lipase activity.

FIG. 18. Alanine scanning to evaluate the relative influence of AV-199-232 (SEQ ID NO: 5) residues on lipase stimulating activity.

FIG. 19. 3D structures of LpL and AV-199-232 were generated by homology-based modeling using Phyre2 followed by molecular refinement at GalaxyWEB.

FIG. 20. Molecular docking of apoC-III to LpL using ClusPro 2.0 (B).

FIG. 21. Molecular docking of AV199-232 to LpL using ClusPRo2.0.

FIG. 22. Molecular modeling prediction of contact residues stabilizing LpL:AV199-232 interactions.

FIG. 23. (A-C) Leukocyte-LpL activity is detectable and responsive to AV199-232 peptide stimulation. (D) AV199-232 stimulates LpL activity in the presence of normal and dyslipidemic plasma.

### DEFINITIONS

As used herein, the following terms are defined to have the following meanings, unless explicitly stated otherwise.

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The term “amino acid analog” refers to a natural or unnatural amino acid where one or more of the C-terminal carboxy group, the N-terminal amino group and side-chain functional group has been chemically blocked, reversibly or irreversibly, or otherwise modified to another functional group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “peptide” refers a short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (synthetic) sequence.

As used herein, the term “mutant peptide” refers to a variant of a peptide having a distinct amino acid sequence from the most common variant occurring in nature, referred to as the “wild-type” sequence. A mutant peptide may be a subsequence of a mutant protein or polypeptide (e.g., a subsequence of a naturally-occurring protein that is not the most common sequence in nature), or may be a peptide that is not a subsequence of a naturally occurring protein or polypeptide. For example, a “mutant ApoA-V peptide” may be a subsequence of a mutant version of ApoA-V or may be distinct sequence not found in naturally-occurring ApoA-V proteins.

As used herein, the term “synthetic peptide” refers to a peptide having a distinct amino acid sequence from those found in natural peptides and/or proteins. A synthetic protein is not a subsequence of a naturally occurring protein, either the wild-type (i.e., most abundant) or mutant versions thereof. For example, a “synthetic Apo-V peptide” (“sAV peptide”) is not a subsequence of naturally occurring ApoA-V. A “synthetic peptide,” as used herein, may be produced or synthesized by any suitable method (e.g., recombinant expression, chemical synthesis, enzymatic synthesis, etc.).

The terms “peptide mimetic” or “peptidomimetic” refer to a peptide-like molecule that emulates a sequence derived from a protein or peptide. A peptide mimetic or peptidomimetic may contain amino acids and/or non-amino acid components. Examples of peptidomimitecs include chemically modified peptides, peptoids (side chains are appended to the nitrogen atom of the peptide backbone, rather than to the α-carbons), β-peptides (amino group bonded to the β carbon rather than the α carbon), etc.

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:

1) Alanine (A) and Glycine (G);

2) Aspartic acid (D) and Glutamic acid (E);

3) Asparagine (N) and Glutamine (Q);

4) Arginine (R) and Lysine (K);

5) Isoleucine (I), Leucine (L), Methionine (M), and Valine (V);

6) Phenylalanine (F), Tyrosine (Y), and Tryptophan (W);

7) Serine (S) and Threonine (T); and

8) Cysteine (C) and Methionine (M).

Naturally occurring residues may be divided into classes based on common side chain properties, for example: polar positive (histidine (H), lysine (K), and arginine (R)); polar negative (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

As used herein, the term “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

As used herein, the term “grouped residues” refers to a set of amino acids within a peptide, polypeptide, or protein that are physically positioned together in three dimensional space. The grouped residues may or may not be sequential in the primary sequence of the peptide, polypeptide, or protein. The residues may be grouped in a globular domain, may be present on the same surface, or may be presented on the same end or side of a secondary structure (e.g., alpha helix) or tertiary structure within a peptide, polypeptide, or protein. In some embodiments, in addition to being physically positioned together, the grouped residues also exhibit some degree of similarity in residue characteristics (e.g., size, polarity charge, etc.).

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

As used herein, the term “effective amount” refers to the amount of a composition (e.g., a synthetic peptide) sufficient to effect beneficial or desired results. An effective amount can be administered in one or more administrations, applications or dosages and is not intended to be limited to a particular formulation or administration route.

As used herein, the terms “administration” and “administering” refer to the act of giving a drug, prodrug, or other agent, or therapeutic treatment (e.g., synthetic peptide) to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) (e.g., multiple synthetic peptide or a synthetic peptide and another therapeutic agent) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “treatment” means an approach to obtaining a beneficial or intended clinical result. The beneficial or intended clinical result may include alleviation of symptoms, a reduction in the severity of the disease, inhibiting a underlying cause of a disease or condition, steadying diseases in a non-advanced state, delaying the progress of a disease, and/or improvement or alleviation of disease conditions.

As used herein, the term “pharmaceutical composition” refers to the combination of an active agent (e.g., synthetic AV peptide) with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintigrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. (1975), incorporated herein by reference in its entirety.

As used herein, the term “constrained alkyl” refers to branched, cyclic, fused alkyl, and adamantyl groups.

As used herein, the term “lower alkyl” refers to C1 to C4 alkyl groups (e.g., methyl, ethyl, propyl, and butyl groups).

Many embodiments herein are described using open “comprising” language. Such embodiments encompass multiple closed “consisting of” and/or “consisting essentially of” embodiments, which may alternatively be claimed or described using such language.

### DETAILED DESCRIPTION

Provided herein are compositions comprising synthetic lipase-stimulating peptides, and methods of treating hypertriglyceridemia and other conditions and diseases therewith. In particular, synthetic peptides (AV-peptides) and peptidomimetics (AV-peptidomimetics) are provided that exhibit the lipase-stimulating activity of apoA-V or an enhancement thereof, as well as methods of use thereof.

Provided herein are synthetic peptides (ApoA-V peptides or, synonomously, AV-peptides), which stimulate the triglyceride (TG) hydrolyzing activities of a subset of lipases including lipoprotein lipase (LpL), endothelial lipase (EL) and hepatic lipase (HL) (e.g., by at least 2-fold (e.g., >2-fold, >3-fold, >4-fold, >5-fold, >6-fold, >7-fold, >8-fold, >9-fold, >10-fold, or more, or ranges therein) with respect to SEQ ID NO: 1, SEQ ID NO: 3, SEQ ID NO: 5, etc.) in one or more of the assays described herein. These lipases are cell surface enzymes that, for example, reduce the concentration of circulating TGs in plasma. Stimulation of the enzymatic activities of these lipases (e.g., LpL, EL, HL, etc.) results in the reduction in plasma TG levels and provides a highly specific therapeutic treatment for clinically elevated TG known as hypertriglyceridemia (HTG).

Triglyceride (also triacylglyceride, TG) is a type of lipid composed of a glycerol and 3-fatty acids that are linked by ester bonds. TG are the main constituents of vegetable oils and animal fats and fatty acids are the most energy dense of all the macronutrients containing about 2.2× more caloric energy than carbohydrates (e.g., glucose and glycogen). In humans, plasma TG concentrations are normally maintained at about 100-150 mg/dL. However, TG levels have been rising steadily over the last 30 years and currently it is estimated that about a third of the population in the industrialized world have chronically elevated plasma TG (>150 mg/dL). This condition, known as hypertriglyceridemia (HTG), can have a number of causes, including lifestyle/dietetics, medications and genetics and is a recognized risk factor for coronary artery disease (CAD) (including ischemic stroke, heart disease), metabolic syndrome and pancreatitis (>500mg/dL) (Do, R., et al., (2013) Nat.Genet. 45, 1345-1352; herein incorporated by reference in its entirety). There are also strong indications that hypertriglyceridemia (HTG) is a risk factor for developing insulin resistance and diabetes, nonalcoholic fatty liver disease (NAFLD) and cancer. Although the exact mechanism is unclear, hypertriglyceridemia (HTG) is associated with an increase in LDL-cholesterol and decrease in HDL-cholesterol, which also contributes to proatherogenic dyslipidemia and likely due to impaired lipoprotein processing. Patients with HTG can be divided into categories of differing severity and the percentage of the population that falls into these categories has been estimated in a number of clinical studies (Do, R., et al., (2013) Nat.Genet. 45, 1345-1352; Ford et al. (2009) Arch.Intern.Med. 169, 572-578; Christian et al. (2011) Am.J.Cardiol. 107, 891-897; herein incorporated by reference in their entireties).

TGs are cleared from circulation primarily through the enzymatic actions of a family of lipases, lipoprotein lipase (LpL), endothelial lipase (EL) and hepatic lipase (HL), which are tethered to the cell surface by high affinity interactions with heparan sulfate. These lipases in turn bind TG-rich lipoproteins (chylomicrons, VLDL, LDL and HDL) in circulation and hydrolyze their TG into free fatty acids and monoacylglycerol, that are readily taken up by cells and metabolized. Lipase enzyme activity is influenced by a number of exchangeable apolipoproteins (apo) associated with lipoproteins. Perhaps the best characterized are apoC-II and apoC-III that stimulate and inhibit lipase activity, respectively.

In some embodiments, provided herein are compositions, kits, systems, and/or methods to treat or prevent one or more of: hypertriglyceridemia, increased triglyceride levels, cardiovascular disease, arthrosclerosis, acute pancreatitis, diabetes, hepatic steatosis, and/or related diseases and conditions. In some embodiments, the hydrolysis of triglycerides is activated by compositions and methods described herein. In some embodiments, triglyceride-hydrolyzing proteins (e.g., lipoprotein lipase (LpL), endothelial lipase (EL) and hepatic lipase (HL)) and/or pathways are activated by the compositions and methods described herein. In some embodiments, compositions and methods are utilized in the treatment and/or prevention of: hypertriglyceridemia, increased triglyceride levels, cardiovascular disease, arthrosclerosis, acute pancreatitis, diabetes, hepatic steatosis, and/or related diseases and conditions. In some embodiments, compositions and methods are utilized in screening for peptides and polypeptides useful in the treatment and/or prevention of: hypertriglyceridemia, increased triglyceride levels, cardiovascular disease, arthrosclerosis, acute pancreatitis, diabetes, hepatic steatosis and/or related diseases and conditions.

In some embodiments, provided herein are pharmaceutical compositions, peptides, proteins, polypeptides, nucleic acids encoding peptides, proteins and polypeptides, molecular complexes of the foregoing, etc. for the treatment or prevention of hypertriglyceridemia, increased triglyceride levels, and/or related diseases and conditions. In some embodiments, provided herein are AV-peptides and polypeptides (e.g., comprising less than 100% sequence identity full length ApoA-V (SEQ ID NO: 1), or a fragment of full length ApoA-V (e.g., SEQ ID NO: 2, SEQ ID NO: 3, SEQ ID NO: 4, SEQ ID NO: 5, SEQ ID NO: 6, SEQ ID NO: 7, SEQ ID NO: 8, or truncated versions thereof), etc.). In some embodiments, a peptide is a synthetic peptide. In some embodiments, a polypeptide or peptide described herein is prepared by methods known to those of ordinary skill in the art. For example, the peptide or polypeptide can be synthesized using solid phase polypeptide synthesis techniques (e.g. Fmoc or Boc chemistry). Alternatively, the peptide or polypeptide can be produced using recombinant DNA technology (e.g., using bacterial or eukaryotic expression systems). Further, a peptide or polypeptide may be expressed within a subject (e.g., following administration of an appropriate vector). Accordingly, to facilitate such methods, provided herein are genetic vectors (e.g., plasmids, viral vectors (e.g. AAV), etc.) comprising a sequence encoding the polypeptide, as well as host cells comprising such vectors. Furthermore, provided herein are the peptides and polypeptides produced via such methods.

In some embodiments, the administration of ApoA-V-based peptides and polypeptides and compositions related thereto (e g mimetics of ApoA-V-based peptides and polypeptides, nucleic acids encoding ApoA-V-based peptides and polypeptides, etc.) is provided. In some embodiments, provided herein is the administration of peptides and polypeptides which enhance triacylglyceride degradation (e.g., hydrolysis, etc.), or are otherwise described herein. Examples of such peptides and polypeptides include those selected from the group consisting of SEQ ID NOS: 2-29. In some embodiments, a peptide or polypeptide is provided comprising or consisting of one or more of SEQ ID NO: 2, SEQ ID NO: 3 SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29. In some embodiments, a peptide or polypeptide is provided comprising at least 50% sequence identity to one of SEQ ID NO: 1, SEQ ID NO: 2, SEQ ID NO: 3 SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 (e.g. at least 60% sequence identity, at least 70% sequence identity, at least 80% sequence identity, at least 90% sequence identity, at least 95% sequence identity, etc.). In some embodiments, peptide and polypeptides comprise at least one mutation from a wild-type sequence (e.g., SEQ ID NOS:1-8). In some embodiments, a peptide or polypeptide comprises:
ARLSRCVQVLSR(X1)LTL(X2)AKAL(X3)ARIQQNLDQLRE, wherein X1, X2, and X3 are independently any amino acid (SEQ ID NO: 15);
ARLSRCVQVLSR(B1)LTL(B2)AKAL(B3)ARIQQNLDQLRE, wherein B1, B2, and B3 are independently any acidic and/or charged amino acid (SEQ ID NO: 16);
ARLSRCVQVLSR(K/Q)LTL(K/Q)AKAL(H/K/Q)ARIQQNLDQLRE, (SEQ ID NO: 17). In some embodiments, provided herein are methods comprising the administration (e.g., to a subject, to cells, to a sample, etc.) of one or more of the aforementioned ApoA-V peptides or polypeptides.

Experiments conducted during development of embodiments of herein demonstrated AV199-232 (SEQ ID NO: 5) to be a minimal portion of ApoA-V (SEQ ID NO: 1) for optimally inducing LpL activity. Experiments, including biochemical mutational analysis, computational studies, molecular modeling, and alanine scanning, indicate various residues of AV199-232 that are: (1) minimally implicated in lipase stimulation, (2) moderately implicated in lipase stimulation, and (3) highly implicated in lipase stimulations. Experiments also indicate certain residues wherein substitutions relative to the natural AV199-232 sequence provides (or may provide) enhancement of one or more characteristics of an AV-peptide (e.g., LpL activation, solubility, lipid binding, heparin binding, etc.). In some embodiments, AV-peptides are provided that comprise the AV199-232 sequence (e.g., SEQ ID NO: 5) with one or more non-natural substitutions.

In some embodiments, an AV-peptide comprises conservative, semi-conservative, and/or non-conservative substitutions relative to SEQ ID NO: 5 at positions that data herein indicate are minimally implicated in lipase activity stimulation. In some embodiments, an AV-peptide comprises conservative and/or semi-conservative substitutions relative to SEQ ID NO: 5 at positions that data herein indicate are minimally implicated in lipase activity stimulation. In some embodiments, an AV-peptide comprises conservative substitutions relative to SEQ ID NO: 5 at positions that data herein indicate are minimally implicated in lipase activity stimulation.

In some embodiments, an AV-peptide comprises conservative, semi-conservative, and/or certain non-conservative substitutions relative to SEQ ID NO: 5 at positions that data herein indicate are moderately implicated in lipase activity stimulation. In some embodiments, an AV-peptide comprises conservative and/or semi-conservative substitutions relative to SEQ ID NO: 5 at positions that data herein indicate are moderately implicated in lipase activity stimulation. In some embodiments, an AV-peptide comprises conservative substitutions relative to SEQ ID NO: 5 at positions that data herein indicate are moderately implicated in lipase activity stimulation.

In some embodiments, an AV-peptide comprises conservative, semi-conservative, and/or no substitutions relative to SEQ ID NO: 5 at positions that data herein indicate are highly implicated in lipase activity stimulation. In some embodiments, an AV-peptide comprises conservative or no substitutions relative to SEQ ID NO: 5 at positions that data herein indicate are highly implicated in lipase activity stimulation. In some embodiments, an AV-peptide comprises no substitutions relative to SEQ ID NO: 5 at positions that data herein indicate are highly implicated in lipase activity stimulation.

In some embodiments, various experiments conducted during development of embodiments herein indicate residues (e g., minimally, moderately, or highly implicated in lipase activity stimulation) in which substitution (e.g., conservative, semi-conservative, non-conservative) to a particular residue or class of residues enhances stimulation of lipase activity. Various such positions within the AV199-232 peptide are apparent from the experiments and data described herein. Certain substitutions that may be particularly beneficial are apparent from SEQ ID NO: 29:

AR(L/F/Y/W)(S/T/N/G)(R/K/hLys/hArg)(C/S/T/N/Q/Y/H)
(V/F/Y/W)QV(L/F/Y/W)S(R/K/hLys/hArg)(K/R/hArg/
hLys/Orn)(L/F/Y/W)TL(K/R/hArg/hLys/Orn)(A/F/Y/W)
(R/K/hLys/hArg)A(L/F/Y/W)(H/K/hLys/R/hArg/Orn)AR
(I/F/Y/W)QQN(L/F/Y/W)DQ(L/F/Y/W)RE;
or
ARX₃X₄X₅X₆X₇QVX₁₀SX₁₂X₁₃X₁₄TLX₁₇X₁₈X₁₉AX₂₁X₂₂ARX₂₅QQNX₂₉
DQX₃₂RE,
wherein: X₃ is L, F, Y, or W; X₄ is S, T, N, or G;
X₅ is R, K, hLys, or hArg; X₆ is C, S, T, N, Q, Y,
or H; X₇ is V, F, Y, or W; X₁₀ is L, F, Y, or W;
X₁₂ is R, K, hLys, or hArg; X₁₃ is K, R, hArg, hLys,
or Orn; X₁₄ is L, F, Y, or W; X₁₇ is K, R, hArg,
hLys, or Orn; X₁₈ is A, F, Y, or W; X₁₉ is R, K,
hLys, or hArg; X₂₁ is L, F, Y, or W; X₂₂ is H, K,
hLys, R, hArg, or Orn; X₂₅ is I, F, Y, or W; X₂₉
is L, F, Y, or W; and X₃₂ is L, F, Y, or W.

In some embodiments, experiments and analysis described herein indicate that the various substitutions allowed for in SEQ ID NO: 29 enhance or do not significantly diminish lipase stimulatory activity of the peptide. Other substitutions may also be indicated by the data described herein, and are within the scope of embodiments herein.

In some embodiments, Table 1 (Example 8) provides substitutions relative to SEQ ID NO: 5 that are within the scope of embodiments herein.

Embodiments are not limited by the specific substitutions described herein. In some embodiments, peptides meeting limitations described herein (e.g., aliphatic helix, LpL stimulation, similar domain organization, etc.) and having substitutions not explicitly described are within the scope of embodiments here. In some embodiments, the peptides described herein are further modified (e.g., substitution, deletion, or addition of standard amino acids; chemical modification; etc.). Modifications that are understood in the field include N-terminal modification, C-terminal modification (which protects the peptide from proteolytic degradation), alkylation of amide groups, hydrocarbon “stapling” (e.g., to stabilize alpha-helix conformations). In some embodiments, the peptides described herein may be modified by conservative residue substitutions, for example, of the charged residues (K to R, R to K, D to E and E to D). In some embodiments, such conservative substitutions provide subtle changes, for example, to the lipases and heparin binding sites with the goal of improving specificity and/or lipase stimulating performance For example, peptides rich R residues tend to bind heparin with higher affinity than peptides rich in K. Modifications of the terminal carboxy group include, without limitation, the amide, lower alkyl amide, constrained alkyls (e.g. branched, cyclic, fused, adamantyl) alkyl, dialkyl amide, and lower alkyl ester modifications. Lower alkyl is C1-C4 alkyl. Furthermore, one or more side groups, or terminal groups, may be protected by protective groups known to the ordinarily-skilled peptide chemist. The α-carbon of an amino acid may be mono- or dimethylated.

In some embodiments, one or more intra-peptide disulfide bonds are introduced (e.g., between two cysteines (e.g., C6 and a substituted cysteine, two substituted cysteines, one or two cysteines outside of the AV peptide sequence, etc.) within the peptide. In some embodiments, the presence of an intra-peptide disulfide bond stabilizes the peptide.

In some embodiments, any embodiments described herein may comprise AV-peptidomimetics corresponding to the AV-peptides described herein with various modifications that are understood in the field. In some embodiments, residues in the peptide sequences described herein may be substituted with amino acids having similar characteristics (e.g., hydrophobic to hydrophobic, neutral to neutral, etc.) or having other desired characteristics (e.g., more acidic, more hydrophobic, less bulky, more bulky, etc.). In some embodiments, non-natural amino acids (or naturally-occurring amino acids other than the standard 20 amino acids) are substituted in order to achieve desired properties.

In some embodiments, residues having a side chain that is positively charged under physiological conditions, or residues where a positively-charged side chain is desired, are substituted with a residue including, but not limited to: lysine, homolysine, δ-hydroxylysine, homoarginine, 2,4-diaminobutyric acid, 3-homoarginine, D-arginine, arginal (—COOH in arginine is replaced by—CHO), 2-amino-3-guanidinopropionic acid, nitroarginine (N(G)-nitroarginine), nitrosoarginine (N(G)-nitrosoarginine), methylarginine (N-methyl-arginine), ε-N-methyllysine, allo-hydroxylysine, 2,3-diaminopropionic acid, 2,2′-diaminopimelic acid, ornithine, sym-dimethylarginine, asym-dimethylarginine, 2,6-diaminohexinic acid, p-aminobenzoic acid and 3-aminotyrosine and, histidine, 1-methylhistidine, and 3-methylhistidine.

A neutral residue is a residue having a side chain that is uncharged under physiological conditions. A polar residue preferably has at least one polar group in the side chain. In some embodiments, polar groups are selected from hydroxyl, sulfhydryl, amine, amide and ester groups or other groups which permit the formation of hydrogen bridges.

In some embodiments, residues having a side chain that is neutral/polar under physiological conditions, or residues where a neutral side chain is desired, are substituted with a residue including, but not limited to: asparagine, cysteine, glutamine, serine, threonine, tyrosine, citrulline, N-methylserine, homoserine, allo-threonine and 3,5-dinitro-tyrosine, and β-homoserine.

Residues having a non-polar, hydrophobic side chain are residues that are uncharged under physiological conditions, preferably with a hydropathy index above 0, particularly above 3. In some embodiments, non-polar, hydrophobic side chains are selected from alkyl, alkylene, alkoxy, alkenoxy, alkylsulfanyl and alkenylsulfanyl residues having from 1 to 10, preferably from 2 to 6, carbon atoms, or aryl residues having from 5 to 12 carbon atoms. In some embodiments, residues having a non-polar, hydrophobic side chain are, or residues where a non-polar, hydrophobic side chain is desired, are substituted with a residue including, but not limited to: leucine, isoleucine, valine, methionine, alanine, phenylalanine, N-methylleucine, tert-butylglycine, octylglycine, cyclohexylalanine, β-alanine, 1-aminocyclohexylcarboxylic acid, N-methylisoleucine, norleucine, norvaline, and N-methylvaline.

In some embodiments, peptide and polypeptides are isolated and/or purified (or substantially isolated and/or substantially purified). Accordingly, in such embodiments, peptides and/or polypeptides are provided in substantially isolated form. In some embodiments, peptides and/or polypeptides are isolated from other peptides and/or polypeptides as a result of solid phase peptide synthesis, for example. Alternatively, peptides and/or polypeptides can be substantially isolated from other proteins after cell lysis from recombinant production. Standard methods of protein purification (e.g., HPLC) can be employed to substantially purify peptides and/or polypeptides. In some embodiments, the present invention provides a preparation of peptides and/or polypeptides in a number of formulations, depending on the desired use. For example, where the polypeptide is substantially isolated (or even nearly completely isolated from other proteins), it can be formulated in a suitable medium solution for storage (e.g., under refrigerated conditions or under frozen conditions). Such preparations may contain protective agents, such as buffers, preservatives, cryprotectants (e.g., sugars such as trehalose), etc. The form of such preparations can be solutions, gels, etc. In some embodiments, ApoA-V peptides and/or polypeptides are prepared in lyophilized form. Moreover, such preparations can include other desired agents, such as small molecules or other peptides, polypeptides or proteins. Indeed, such a preparation comprising a mixture of different embodiments of the peptides and/or polypeptides described here may be provided.

In some embodiments, provided herein are peptidomimetic versions of the peptide sequences described herein or variants thereof In some embodiments, a peptidomimetic is characterized by an entity that retains the polarity (or non-polarity, hydrophobicity, etc.), three-dimensional size, and functionality (bioactivity) of its peptide equivalent but wherein all or a portion of the peptide bonds have been replaced (e.g., by more stable linkages). In some embodiments, ‘stable’ refers to being more resistant to chemical degradation or enzymatic degradation by hydrolytic enzymes. In some embodiments, the bond which replaces the amide bond (e.g., amide bond surrogate) conserves some properties of the amide bond (e.g., conformation, steric bulk, electrostatic character, capacity for hydrogen bonding, etc.). Chapter 14 of “Drug Design and Development”, Krogsgaard, Larsen, Liljefors and Madsen (Eds) 1996, Horwood Acad. Publishers provides a general discussion of techniques for the design and synthesis of peptidomimetics and is herein incorporated by reference in its entirety. Suitable amide bond surrogates include, but are not limited to: N-alkylation (Schmidt, R. et al., Int. J. Peptide Protein Res., 1995, 46,47; herein incorporated by reference in its entirety), retro-inverse amide (Chorev, M. and Goodman, M., Acc. Chem. Res, 1993, 26, 266; herein incorporated by reference in its entirety), thioamide (Sherman D. B. and Spatola, A. F. J. Am. Chem. Soc., 1990, 112, 433; herein incorporated by reference in its entirety), thioester, phosphonate, ketomethylene (Hoffman, R. V. and Kim, H. O. J. Org. Chem., 1995, 60, 5107; herein incorporated by reference in its entirety), hydroxymethylene, fluorovinyl (Allmendinger, T. et al., Tetrahydron Lett., 1990, 31, 7297; herein incorporated by reference in its entirety), vinyl, methyleneamino (Sasaki, Y and Abe, J. Chem. Pharm. Bull. 1997 45, 13; herein incorporated by reference in its entirety), methylenethio (Spatola, A. F., Methods Neurosci, 1993, 13, 19; herein incorporated by reference in its entirety), alkane (Lavielle, S. et. al., Int. J.Peptide Protein Res., 1993, 42, 270; herein incorporated by reference in its entirety) and sulfonamido (Luisi, G. et al. Tetrahedron Lett. 1993, 34, 2391; herein incorporated by reference in its entirety).

As well as replacement of amide bonds, peptidomimetics may involve the replacement of larger structural moieties with di- or tripeptidomimetic structures and in this case, mimetic moieties involving the peptide bond, such as azole-derived mimetics may be used as dipeptide replacements. Suitable peptidomimetics include reduced peptides where the amide bond has been reduced to a methylene amine by treatment with a reducing agent (e.g. borane or a hydride reagent such as lithium aluminum-hydride); such a reduction has the added advantage of increasing the overall cationicity of the molecule.

Other peptidomimetics include peptoids formed, for example, by the stepwise synthesis of amide-functionalised polyglycines. Some peptidomimetic backbones will be readily available from their peptide precursors, such as peptides which have been permethylated, suitable methods are described by Ostresh, J. M. et al. in Proc. Natl. Acad. Sci. USA (1994) 91, 11138-11142; herein incorporated by reference in its entirety.

In some embodiments, peptides are treated or conditioned prior to use. For example, ApoA-V peptides may be incubated with very low density lipoproteins (VLDL). In some embodiments, AV-peptides may be incubated with heparin, synthetic lipoproteins, liposomes or a patient's own lipoproteins to enhance bioavailability and therapeutic efficacy.

In some embodiments, the peptides described herein are provided as fusions with other peptides or polypeptides. Such fusions may be expressed from a recombinant DNA which encodes the ApoA-V peptide and the additional peptide/polypeptide or may be formed by chemical synthesis. For instance, the fusion may comprise a ApoA-V peptide and an enzyme of interest, a luciferase, RNasin or RNase, and/or a channel protein (e.g., ion channel protein), a receptor, a membrane protein, a cytosolic protein, a nuclear protein, a structural protein, a phosphoprotein, a kinase, a signaling protein, a metabolic protein, a mitochondrial protein, a receptor associated protein, a fluorescent protein, an enzyme substrate, a transcription factor, selectable marker protein, nucleic acid binding protein, extracellular matrix protein, secreted protein, receptor ligand, serum protein, a protein with reactive cysteines, a transporter protein, a targeting sequence (e.g., a myristylation sequence), a mitochondrial localization sequence, or a nuclear localization sequence. The additional peptide/polypeptide may be fused to the N-terminus and/or the C-terminus of the ApoA-V peptide. In one embodiment, the fusion protein comprises a first peptide/polypeptide at the N-terminus and another (different) peptide/polypeptide at the C-terminus of the ApoA-V peptide. Optionally, the elements in the fusion are separated by a connector sequence, e.g., preferably one having at least 2 amino acid residues, such as one having 13 and up to 40 or 50 amino acid residues. The presence of a connector sequence in a fusion protein of the invention does not substantially alter the function of either element (e.g., the ApoA-V peptide) in the fusion relative to the function of each individual element, likely due to the connector sequence providing flexibility (autonomy) for each element in the fusion. In certain embodiment, the connector sequence is a sequence recognized by an enzyme or is photocleavable. For example, the connector sequence may include a protease recognition site.

In some embodiments, provided herein are pharmaceutical compositions comprising of one or more AV-peptides and polypeptides described herein (e.g., having less than 100% sequence identity with SEQ ID NO: 5, exhibiting enhanced activation of triglyceride-hydrolyzing lipases, etc.) and a pharmaceutically acceptable carrier. Any carrier which can supply an active peptide or polypeptide (e.g., without destroying the peptide or polypeptide within the carrier) is a suitable carrier, and such carriers are well known in the art. In some embodiments, compositions are formulated for administration by any suitable route, including but not limited to, orally (e.g., such as in the form of tablets, capsules, granules or powders), sublingually, bucally, parenterally (such as by subcutaneous, intravenous, intramuscular, intradermal, or intrasternal injection or infusion (e.g., as sterile injectable aqueous or non-aqueous solutions or suspensions, etc.)), nasally (including administration to the nasal membranes, such as by inhalation spray), topically (such as in the form of a cream or ointment), transdermally (such as by transdermal patch), rectally (such as in the form of suppositories), etc.

In some embodiments, provided herein are methods for treating patients suffering from (or at risk of) hypertriglyceridemia and/or in need of treatment (or preventative therapy). In some embodiments, a pharmaceutical composition comprising at least one AV-peptide or polypeptide described herein is delivered to such a patient in an amount and at a location sufficient to treat the condition. In some embodiments, peptides and/or polypeptides (or pharmaceutical composition comprising such) can be delivered to the patient systemically or locally, and it will be within the ordinary skill of the medical professional treating such patient to ascertain the most appropriate delivery route, time course, and dosage for treatment. It will be appreciated that application methods of treating a patient most preferably substantially alleviates or even eliminates such symptoms; however, as with many medical treatments, application of the inventive method is deemed successful if, during, following, or otherwise as a result of the inventive method, the symptoms of the disease or disorder in the patient subside to an ascertainable degree.

A pharmaceutical composition may be administered in the form which is formulated with a pharmaceutically acceptable carrier and optional excipients, adjuvants, etc. in accordance with good pharmaceutical practice. The AV-peptide-based pharmaceutical composition may be in the form of a solid, semi-solid or liquid dosage form: such as powder, solution, elixir, syrup, suspension, cream, drops, paste and spray. As those skilled in the art would recognize, depending on the chosen route of administration (e.g. pill, injection, etc.), the composition form is determined. In general, it is preferred to use a unit dosage form in order to achieve an easy and accurate administration of the active pharmaceutical peptide or polypeptide. In general, the therapeutically effective pharmaceutical compound is present in such a dosage form at a concentration level ranging from about 0.5% to about 99% by weight of the total composition, e.g., in an amount sufficient to provide the desired unit dose. In some embodiments, the pharmaceutical composition may be administered in single or multiple doses. The particular route of administration and the dosage regimen will be determined by one of skill in keeping with the condition of the individual to be treated and said individual's response to the treatment. In some embodiments, an AV-peptides-based pharmaceutical composition is provided in a unit dosage form for administration to a subject, comprising a AV-peptides or polypeptide (e.g., comprising less than 100% sequence identity with a portion of SEQ ID NO: 2, etc.) and one or more nontoxic pharmaceutically acceptable carriers, adjuvants or vehicles. The amount of the active ingredient that may be combined with such materials to produce a single dosage form will vary depending upon various factors, as indicated above. A variety of materials can be used as carriers, adjuvants and vehicles in the composition of the invention, as available in the pharmaceutical art. Injectable preparations, such as oleaginous solutions, suspensions or emulsions, may be formulated as known in the art, using suitable dispersing or wetting agents and suspending agents, as needed. The sterile injectable preparation may employ a nontoxic parenterally acceptable diluent or solvent such as sterile nonpyrogenic water or 1,3-butanediol. Among the other acceptable vehicles and solvents that may be employed are 5% dextrose injection, Ringer's injection and isotonic sodium chloride injection (as described in the USP/NF). In addition, sterile, fixed oils may be conventionally employed as solvents or suspending media. For this purpose, any bland fixed oil may be used, including synthetic mono-, di- or triglycerides. Fatty acids such as oleic acid can also be used in the preparation of injectable compositions. The peptides and the polypeptides encompassing a substantially alpha helical peptide region that are disclosed herein may be further derivatized by chemical alterations, such as amidation, glycosylation, acylation, sulfation, phosphorylation, acetylation, and cyclization. Such chemical alterations can be imparted through chemical or biochemical methodologies, as well as through in vivo processes, or any combination thereof.

In certain embodiments, the peptides and polypeptides described herein are derivatized by modification of the terminal amino group. Such modifications include, without limitation, the desamino, N-lower alkyl, N-di-lower alkyl, constrained alkyls (e.g. branched, cyclic, fused, adamantyl) and N-acyl modifications where the acyl moiety is C6-C20 alkyl.

In certain embodiments, the peptides and polypeptides described herein are derivatized by modification of the terminal carboxyl group. Such modifications include, without limitation, amide, lower alkyl amide, constrained alkyls (e.g. branched, cyclic, fused, adamantyl) alkyl, dialkyl amide, and lower alkyl ester modifications, where lower alkyl is C1-C4 alkyl. Furthermore, one or more side groups, or terminal groups, may be protected by protective groups known to the ordinarily-skilled peptide chemist. The α-carbon of an amino acid may be mono- or dimethylated.

In various embodiments, the peptides and polypeptides disclosed herein are derivatized by conjugation to one or more polymers or small molecule substituents.

In certain of these embodiments, the synthetic peptides and polypeptides described herein are derivatized by coupling to polyethylene glycol (PEG). Coupling may be performed using known processes. See, Int. J. Hematology, 68:1 (1998); Bioconjugate Chem., 6:150 (1995); and Crit. Rev. Therap. Drug Carrier Sys., 9:249 (1992) all of which are incorporated herein by reference in their entirety. Those skilled in the art, therefore, will be able to utilize such well-known techniques for linking one or more polyethylene glycol polymers to the peptides and polypeptides described herein. Suitable polyethylene glycol polymers typically are commercially available or may be made by techniques well known to those skilled in the art. The polyethylene glycol polymers preferably have molecular weights between 500 and 20,000 and may be branched or straight chain polymers.

The attachment of a PEG to a peptide or polypeptide described herein can be accomplished by coupling to amino, carboxyl or thiol groups. These groups will typically be the N- and C-termini and on the side chains of such naturally occurring amino acids as lysine, aspartic acid, glutamic acid and cysteine. Since the peptides and polypeptides of the present disclosure can be prepared by solid phase peptide chemistry techniques, a variety of moieties containing diamino and dicarboxylic groups with orthogonal protecting groups can be introduced for conjugation to PEG.

The present disclosure also provides for conjugation of the peptides and polypeptides described herein to one or more polymers other than polyethylene glycol.

In some embodiments, the peptides and polypeptides described herein are derivatized by conjugation or linkage to, or attachment of, polyamino acids (e.g., poly-his, poly-arg, poly-lys, etc.) and/or fatty acid chains of various lengths to the N- or C-terminus or amino acid residue side chains. In certain embodiments, the peptides and polypeptides described herein are derivatized by the addition of polyamide chains, particularly polyamide chains of precise lengths, as described in U.S. Pat. No. 6,552,167, which is incorporated by reference in its entirety. In yet other embodiments, the peptides and polypeptides are modified by the addition of alkylPEG moieties as described in U.S. Pat. Nos. 5,359,030 and 5,681,811, which are incorporated by reference in their entireties.

In select embodiments, the peptides and polypeptides disclosed herein are derivatized by conjugation to polymers that include albumin and gelatin. See, Gombotz and Pettit, Bioconjugate Chem., 6:332-351, 1995, which is incorporated herein by reference in its entirety.

In further embodiments, the peptides and polypeptides disclosed herein are conjugated or fused to immunoglobulins or immunoglobulin fragments, such as antibody Fc regions.

In various embodiments, the peptides and polypeptides described herein are derivatized by attaching small molecule substituents, including short chain alkyls and constrained alkyls (e.g., branched, cyclic, fused, adamantyl groups), and aromatic groups.

In certain embodiments, the peptides and polypeptides described herein comprise an alkylglycine amino acid analog comprising a C5-C9 straight or branched alkyl side chain, or a cycloalkyl group. In one embodiment, the peptide or polypeptide comprises an alkylglycine comprising a C6-C8 straight or branched alkyl side chain. In another embodiment, the polypeptide comprises an octylglycine comprising a C8 straight alkyl side chain (octyl-glycine).

The peptides and polypeptides described herein may be prepared as salts with various inorganic and organic acids and bases. Such salts include salts prepared with organic and inorganic acids, for example, with HCl, HBr, H₂SO₄, H₃PO₄, trifluoroacetic acid, acetic acid, formic acid, methanesulfonic acid, toluenesulfonic acid, maleic acid, fumaric acid and camphorsulfonic acid. Salts prepared with bases include ammonium salts, alkali metal salts, e.g. sodium and potassium salts, alkali earth salts, e.g. calcium and magnesium salts, and zinc salts. The salts may be formed by conventional means, such as by reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

The peptides and polypeptides described herein can be formulated as pharmaceutically acceptable salts and/or complexes thereof. Pharmaceutically acceptable salts include acid addition salts such as those containing sulfate, hydrochloride, phosphate, sulfamate, acetate, citrate, lactate, tartrate, succinate, oxalate, methanesulfonate, ethanesulfonate, benzenesulfonate, p-toluenesulfonate, cyclohexylsulfamate and quinate. Pharmaceutically acceptable salts can be obtained from acids such as hydrochloric acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quinic acid. Such salts may be prepared by, for example, reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

The peptides and polypeptides described herein may be formulated as pharmaceutical compositions for use in conjunction with the methods of the present disclosure. Compositions disclosed herein may conveniently be provided in the form of formulations suitable for parenteral administration, including subcutaneous, intramuscular and intravenous administration, nasal administration, pulmonary administration, or oral administration. Suitable formulation of peptides and polypeptides for each such route of administration is described in standard formulation treatises, e.g., Remington's Pharmaceutical Sciences by E. W. Martin. See also Wang, Y. J. and Hanson, M. A. “Parenteral Formulations of Proteins and Peptides: Stability and Stabilizers,” Journal of Parenteral Science and Technology, Technical Report No. 10, Supp. 42:2S (1988).

Certain of the peptides and polypeptides described herein may be substantially insoluble in water and sparingly soluble in most pharmaceutically acceptable protic solvents and in vegetable oils. In certain embodiments, cyclodextrins may be added as aqueous solubility enhancers. Cyclodextrins include methyl, dimethyl, hydroxypropyl, hydroxyethyl, glucosyl, maltosyl and maltotriosyl derivatives of alpha-, beta-, and gamma-cyclodextrin. An exemplary cyclodextrin solubility enhancer is hydroxypropyl-beta-cyclodextrin (HPBCD), which may be added to any of the above-described compositions to further improve the aqueous solubility characteristics of the peptides or polypeptides. In one embodiment, the composition comprises 0.1% to 20% HPBCD, 1% to 15% HPBCD, or from 2.5% to 10% HPBCD. The amount of solubility enhancer employed will depend on the amount of peptide or polypeptide of the present disclosure in the composition. In certain embodiments, the peptides may be formulated in non-aqueous polar aprotic solvents such as DMSO, dimethylformamide (DMF) or N-methylpyrrolidone (NMP).

In some cases, it will be convenient to provide the peptide or polypeptide and another active agent in a single composition or solution for administration together. In other cases, it may be more advantageous to administer the additional agent separately from said polypeptide.

For use, pharmaceutical compositions of the peptides and polypeptides described herein may be provided in unit dosage form containing an amount of the peptide or polypeptide effective for a single administration. Unit dosage forms useful for subcutaneous administration include prefilled syringes and injectors.

In some embodiments, AV-peptides/polypeptides are provided in pharmaceutical compositions and/or co-administered (concurrently or in series) with one or more additional therapeutic agents. Such additional agents may be for further reduction of triglyceride levels or for the treatment of related issues (e.g., elevated cholesterol levels, high blood pressure, etc.) conditions or diseases. Additional agents may include, but are not limited to: fabric acid derivatives, niacin, omega-3 fatty acids, hormone drugs (e.g., metreleptin (MYALEPT)), statins (e.g., simvastatin, atorvastatin, rosuvastatin, etc.), antiplatelet agents (e.g., aspirin, clopidogrel, dipyridamole, ticlopidine, etc.), anticoagulants (e.g., warfarin (Coumadin) and heparin), medications for treatment of diabetes (e.g., sulfonylureas biguanides, meglitinides, thiazolidinediones, DPP-4 inhibitors, SGLT2 Inhibitors, alpha-glucosidase inhibitors, bile acid sequestrants, etc.), insulin, GLP-1 peptides (e.g., VICTOZA), etc.

In some embodiments, AV-peptide-based compositions described herein are provided as part of a kit. In some embodiments, a kit of the present invention comprises one or more ApoA-V-based peptides or polypeptides. In some embodiments, a kit comprises an AV-peptide-based composition configured for co-administration with one or more additional compositions (e.g. pharmaceutical compositions). In some embodiments, one or more AV-peptide-based compositions are co-administered with one or more other agents for effective enhancement of reduction of triglycerides or the treatment or prevention of hypertriglyceridemia or a related disease or condition.

In some embodiments, AV-peptide-based compositions are provided for the reduction of triglyceride concentrations, enhancement of degradation (e.g., hydrolysis), and/or treatment or prevention of hypertriglyceridemia, cardiovascular disease, arthrosclerosis, acute pancreatitis, diabetes, hepatic steatosis and/or related diseases and conditions. Various modification, recombination, and variation of the described features and embodiments will be apparent to those skilled in the art without departing from the scope and spirit of the invention. Although specific embodiments have been described, it should be understood that the invention as claimed should not be unduly limited to such specific embodiments. Indeed, various modifications of the described modes and embodiments that are obvious to those skilled in the relevant fields are intended to be within the scope of the following claims.

As addressed herein, in some embodiments, methods of treating hypertriglyceridemia are provided. The methods comprise administering a peptide or polypeptide as described herein to a patient with hypertriglyceridemia in an amount, on a schedule, and for a duration sufficient to reduce blood triglyceride levels.

In certain embodiments, the patient has extreme hypertriglyceridemia, defined as non-fasting plasma triglyceride (“TG”) levels prior to the treatment provided herein ≧885 mg/dL. In some embodiments, the patient has severe hypertriglyceridemia, defined as pre-treatment TG levels between 500 and 885 mg/dL. In some embodiments, the patient has refractory hypertriglyceridemia prior to treatment, defined as TG levels between 200 mg/dL and 500 mg/dL. In other embodiments, the patient prior to treatment has borderline hypertriglyceridemia, between 150 mg/dL and 200 mg/dL. In routine embodiments, the patient will have hypertriglyceridemia despite having been treated with one or more other therapeutic agents prior to initiating the treatment provided herein.

In certain embodiments, the patient will have chylomicronemia. In some embodiments, the patient will have above-normal LDL-c levels. In some embodiments, the patient will have below-normal levels of HDL-c.

In some embodiments, the patient will have pancreatitis. In some embodiments, the patient will have diabetes. In certain embodiments, the patient will have metabolic syndrome. Some patients will have hepatosplenomegaly. Some patients will have non-alcoholic steatohepatitis (“NASH”). Some patients will have lipodystrophy (e.g., congenital, acquired, generalized, partial, etc.)

In certain embodiments, the patient is an adult. In other embodiments, the patient is a child.

In various embodiments, the polypeptide is administered in an amount, on a schedule, and for a duration sufficient to decrease triglyceride levels by at least 5%, 10%, 15%, 20% or 25% or more as compared to levels just prior to initiation of treatment. In some embodiments, the polypeptide is administered in an amount, on a dosage schedule, and for a duration sufficient to decrease triglyceride levels by at least 30%, 35%, 40%, 45% or 50%. In particular embodiments, the polypeptide is administered in an amount, on a schedule, and for a time sufficient to reduce triglyceride levels by at least 55%, 60%, 65%, even at least about 70% or more.

In certain embodiments, the polypeptide is administered in an amount, expressed as a daily equivalent dose regardless of dosing frequency, of 50 micrograms (“mcg”) per day, 60 mcg per day, 70 mcg per day, 75 mcg per day, 100 mcg per day, 150 mcg per day, 200 mcg per day, or 250 mcg per day. In some embodiments, the polypeptide is administered in an amount of 500 mcg per day, 750 mcg per day, or 1 milligram (“mg”) per day. In yet further embodiments, the polypeptide is administered in an amount, expressed as a daily equivalent dose regardless of dosing frequency, of 1-10 mg per day, including 1 mg per day, 1.5 mg per day, 1.75 mg per day, 2 mg per day, 2.5 mg per day, 3 mg per day, 3.5 mg per day, 4 mg per day, 4.5 mg per day, 5 mg per day, 5.5 mg per day, 6 mg per day, 6.5 mg per day, 7 mg per day, 7.5 mg per day, 8 mg per day, 8.5 mg per day, 9 mg per day, 9.5 mg per day, or 10 mg per day.

In various embodiments, the polypeptide is administered on a monthly dosage schedule. In other embodiments, the polypeptide is administered biweekly. In yet other embodiments, the polypeptide is administered weekly. In certain embodiments, the polypeptide is administered daily (“QD”). In select embodiments, the polypeptide is administered twice a day (“BID”).

In typical embodiments, the polypeptide is administered for at least 3 months, at least 6 months, at least 12 months, or more. In some embodiments, the polypeptide is administered for at least 18 months, 2 years, 3 years, or more.

In typical embodiments, the polypeptide is administered parenterally. In some parenteral embodiments, the polypeptide is administered subcutaneously. In some parenteral embodiments, the polypeptide is administered intramuscularly. In yet other embodiments, the polypeptide is administered intravenously. In certain embodiments, the polypeptide is administered by subdermally implanted osmotic minipump (Intarcia).

In yet other embodiments, the polypeptide is administered intranasally. In certain embodiments, the polypeptide is administered by inhalation. In yet other embodiments, the polypeptide is administered orally, by buccal administration, or by sublingual administration.

In other embodiments, the polypeptide is administered transdermally.

In certain preferred embodiments, the polypeptide is administered by subcutaneous injection, once per day, in an amount of from 0.5-5.0 mg, for at least 6 months.

In typical embodiments, the polypeptide is administered as an adjunct to a diet designed to reduce triglyceride levels.

In various embodiments, the polypeptide is administered in combination with at least one additional therapeutic agent. Typically in such embodiments, the additional therapeutic agent is administered as a separate composition. In certain of these embodiments, the additional therapeutic agent is administered by a different route of administration and/or on a different dosage schedule from the polypeptide described herein.

In certain of the combination embodiments, at least one of the at least one additional agent is selected from the group consisting of: niacin; omega-3 polyunsaturated fatty acids; fibrates; mipomersen; and alipogene tiparvovec. In particular embodiments, the omega-3 polyunsaturated fatty acids are selected from the group consisting of icosapent ethyl; omega-3 carboxylic acids; and omega-3-acid ethyl esters. In certain embodiments, at least of the at least one additional agent is a statin. In certain embodiments, the statin is selected from the group consisting of: pravastatin, lovastatin, simvastatin, atorvastatin, fluvastatin, rosuvastatin, tenivastatin, and pitavastatin.

### Experimental

### EXAMPLE 1

### Lipase Stimulating Domain in apoA-V

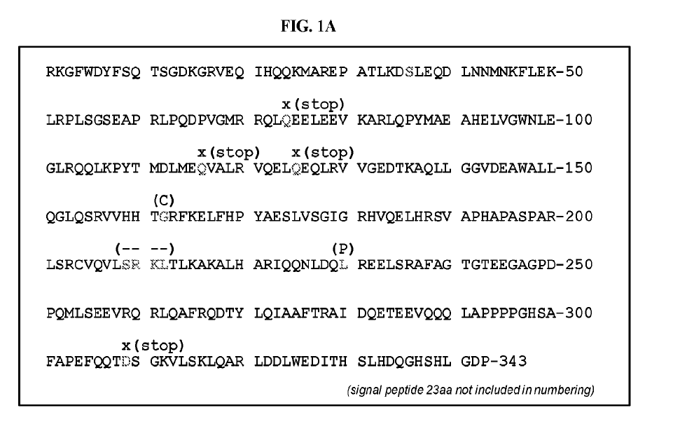

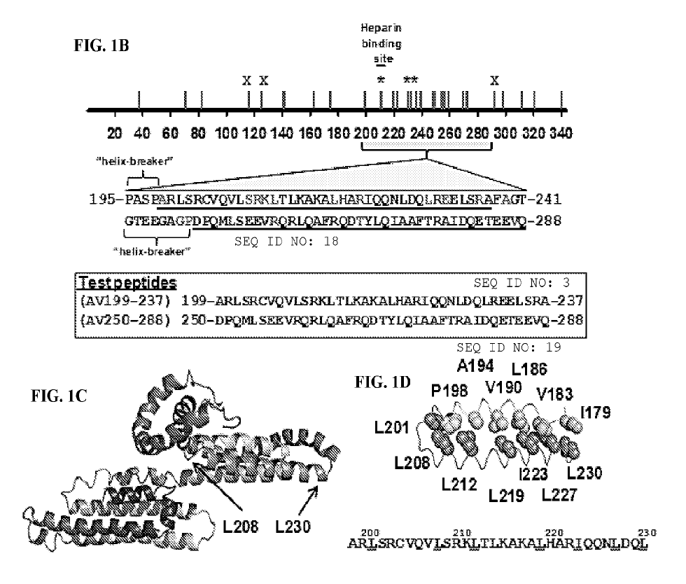

ApoA-V is a relatively large protein at 343 amino acids and three activities have been ascribed to it, lipid binding, heparin/receptor binding and lipases activation (FIG. 1). Of particular interest to us is/are the domain(s) in apoA-V that stimulate lipase activity and a number of mutation identified in human populations have helped to elucidate its location. An important study by Mendoza-Barbera and colleagues (Sharma et al. (2014) Arterioscler. Thromb. Vasc. Biol. 34, 2254-2260; herein incorporated by reference in its entirety) recently characterizing three unrelated APOA5 mutation in patients with HTG identified a (Ser232-Leu235)del and Leu253Pro that significantly impairs apoA-V's lipase stimulating activity. This deletion corresponded to a previously reported 42 amino acid sequence enriched in positively charged residues with strong heparan sulfate binding potential and shown to be necessary for cell surface binding (Nilsson et al. (2007) Biochemistry 46, 3896-3904; herein incorporated by reference in its entirety). The Pro residue (in Leu253Pro), while replacing only one residue position is considered a protein structure disruptor and would likely introduce a significant change in protein conformation affecting the surrounding area. This region also corresponds with an older report showing that recombinant apoA-V in which residues 192-238 were deleted had a diminished ability to bind lipid and activate lipase activity (Sun et al. (2006) Chem. Phys. Lipids 143, 22-28; herein incorporated by reference in its entirety).

Mapping all known sequence variants of apoA-V linked to hypertriglyceridemia and/or CAD reveals that mutations cluster within residues 199-259 (FIG. 1B). A 3D model for apoA-V was deduced with and predicts a structure rich in α-helix (FIG. 1C). Residues 199-259 appear to be organized into two separate α-helixes connected by a short loop. Subsequent experiments were designed based, in part, organization, for example, the synthesis and testing of two peptides spanning residues 199-237 (AV199-237) and residues 250-288 (AV250-288).

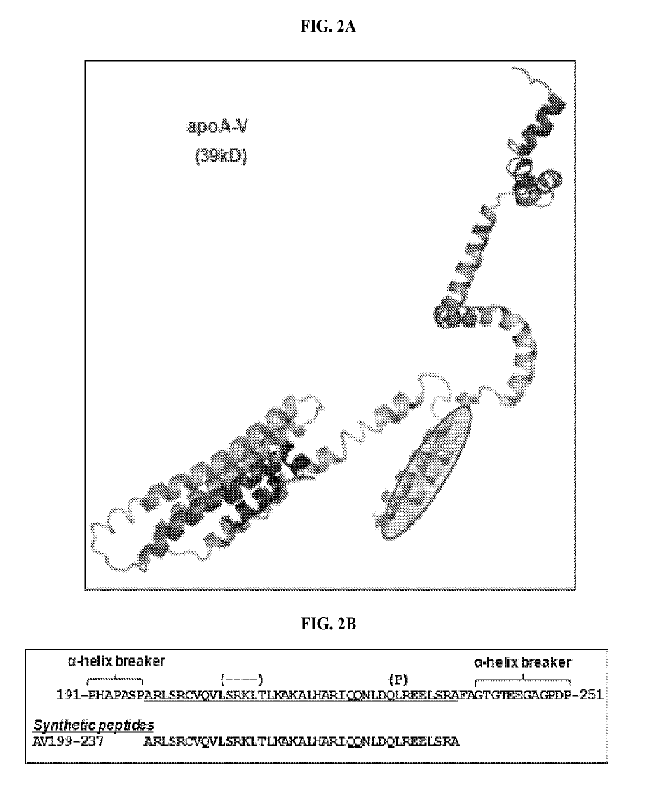

The 3D structure of apoA-V has not been solved, so in experiments conducted during development of embodiments described herein, a wed based server (Phyre2) was used to predict the 3D structure which showed that residues 200-245 appeared to be folded into an α-helix and not a random unfolded structure (FIG. 2). Inspection of the sequence revealed that this domain was separated from the rest of the protein sequence by helix breaker residues (P, G) and demarked the ends of the α-helix.

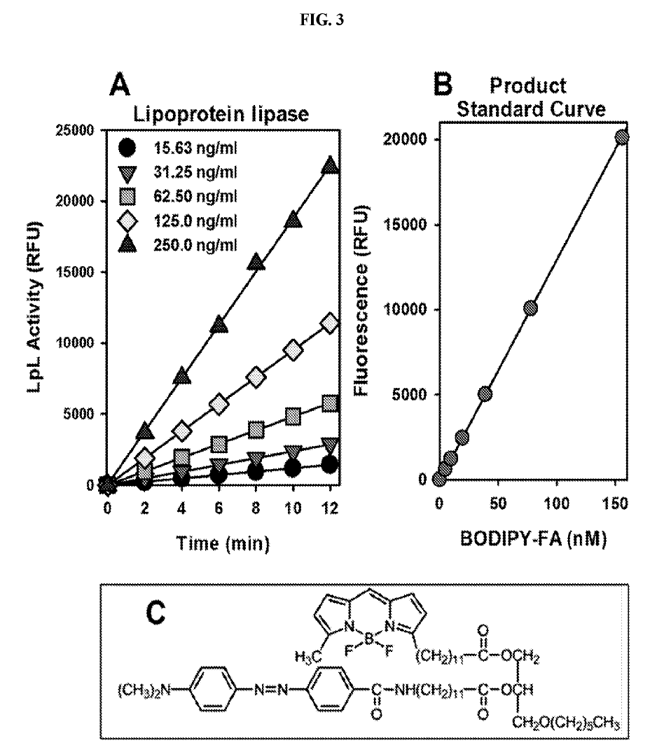

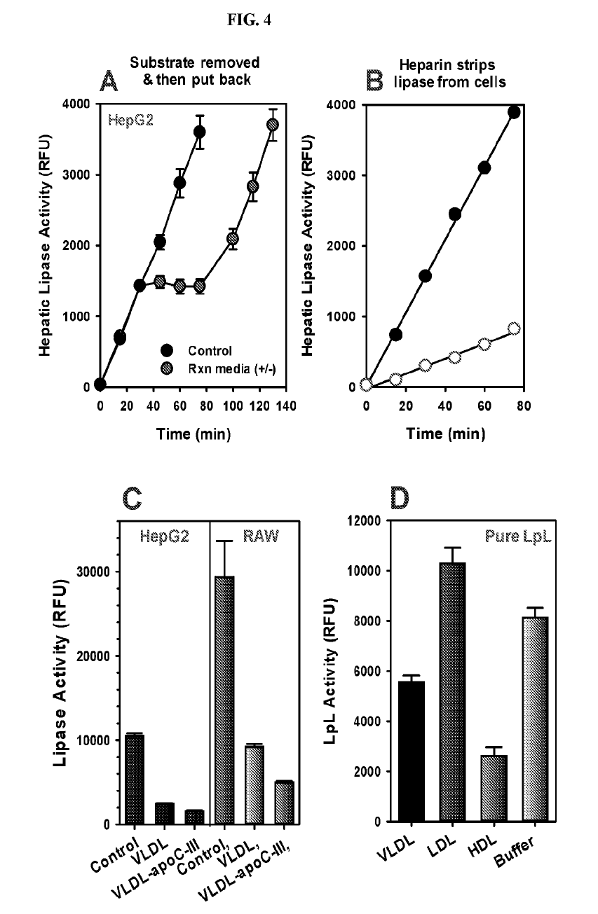

To search for lipase stimulators, a lipase assay was employed using a commercially available fluorogenic-TG substrate (EnzChek) supplied by Molecular Probes (FIG. 3). The protocol was adapted from Basu et al., (Basu et al. (2011) J.Lipid Res. 52, 826-832; herein incorporated by reference in its entirety) with minor modifications in pH and detergent. For in vitro assays, LpL (SIGMA) was diluted to 0.12 μg/ml in 20 mM Tris-HCl, 150 mM NaCl, 1% BSA, 0.015% Zwittergent 3-14, pH 7.8, 1 μM EnzChek substrate, prepared in 96-well black microtiter plates. For some assays the plates were incubates at 37° C. for 10min. Detection was performed in a Synergy H1 Multi-Mode Reader using excitation/emission wavelengths of 482 nm and 515 nm, respectively. Assays showed that the assay was sensitive to nmol/L LpL and linear with increasing enzyme concentrations indicating that the substrate concentration was not limiting. Moreover, the product produced could be quantified using BODIPY-C12 fatty acids to determine the molar rates of enzyme reaction. Since the assay uses a non-toxic substrate and a low amount of neutral detergent it is possible to use this assay with live cells (FIG. 4). The lipase activities assayed were sensitive to both the removal of lipase (HL) from the surface of hepatocytes (by a heparin pre-wash) and to apolipoproteins present in isolated lipoproteins, VLDL, LDL and HDL preparations.

### EXAMPLE 2

### Synthetic Peptides

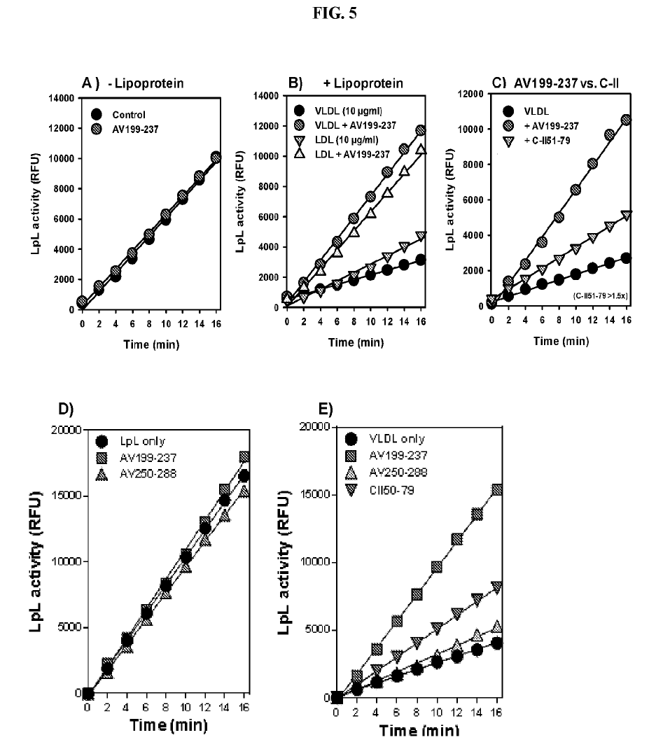

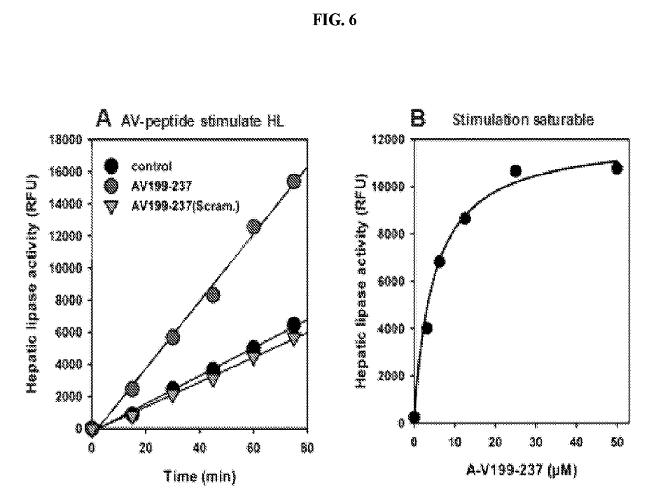

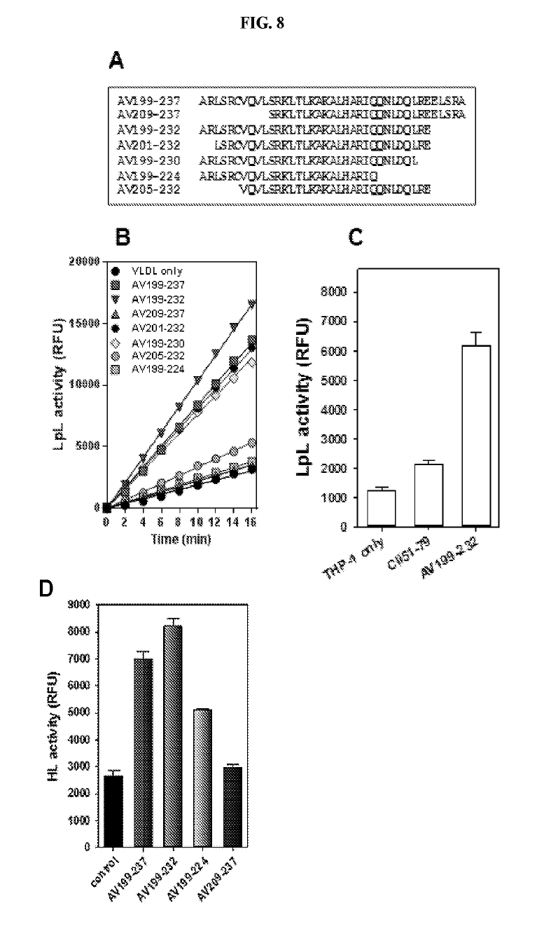

The first peptide synthesized (FIG. 2B) and tested for its influence on LpL activity (FIG. 5A-B) was AV199-237. Initially, when the peptide was added to LpL no change in activity was observed; however, upon incubating the peptide with very low density lipoprotein (VLDL) before adding to LpL, hydrolytic activity was increased ˜4-fold. Little or no stimulating activity was observed for AV250-288, in the presence (FIG. 5E) or absence (FIG. 5D) of VLDL. However, in live cell assays the lipase stimulating activity of AV-peptides does not require VLDL suggesting that the peptide is acting directly on the lipase (FIGS. 6 and 8). This increase in activity with AV199-237 exceeded that seen with an apoC-II peptide (C-II50-79) previously reported to also increase LpL activity. At high concentrations apoC-II has also been reported to inhibit lipase activity.

AV199-237 was also very effective in stimulating lipase activity in cell culture (FIG. 6). Hepatic lipase (HL) was increased about 3-fold and the effect was saturable consistent with the peptide acting on a specific and finite set of lipases. This observation is also unique since LpL and HL are distinct lipases suggesting that unlike apoC-II, which has not influence on HL, apoA-V and its peptidomimetics can stimulate both LpL and HL. Also of note is that the stimulation of HL in cells culture was sustained for at least 75 min (assay stopped).

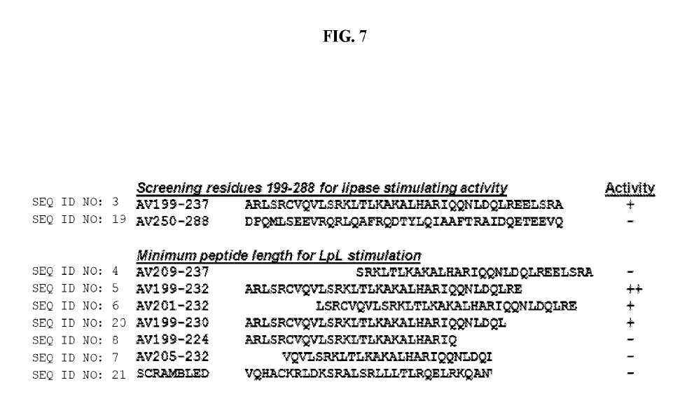

To determine if the lipase stimulating function could be localized to a shorter peptide sequence, a series of shortened peptides were generated to test in the lipase assays (FIG. 7B, 7D). The testing performed in both in LpL in vitro and cell culture assays revealed that the AV199-237 could be shortened by 5-residues and the resulting AV199-232 peptide harbored full activity and was actually somewhat more potent (FIG. 8).

### EXAMPLE 3

### LpL Enzyme Kinetics and AV199-232

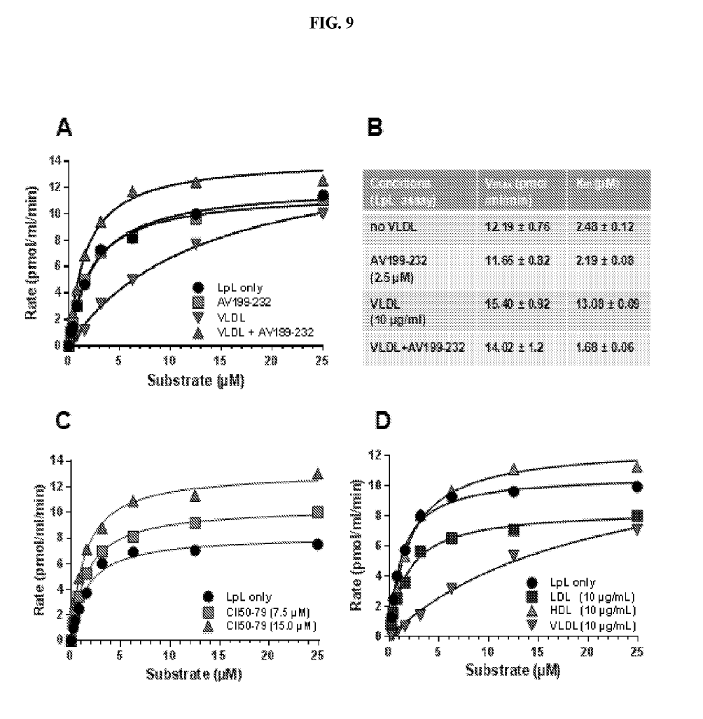

The influence of AV199-232 on the kinetic parameters of LpL activity (e.g., Vmax and Km) was investigated (FIG. 9A-B). The addition of VLDL was observed to reduce LpL affinity for substrate (Km increase) by about 5-fold. This was due to the apoC-III content of VLDL. However, the addition of AV199-232 (2.5 μM) substantially improved the Km by almost 8-fold indicating that the AV199-232 is likely binding to LpL and stimulating its active site allosterically. Also AV199-232 was functional in the presence of apoC-III in VLDL. The effect of different lipoproteins on LpL kinetics was compared (FIG. 9D). Neither HDL or LDL had a significant effect on Km.

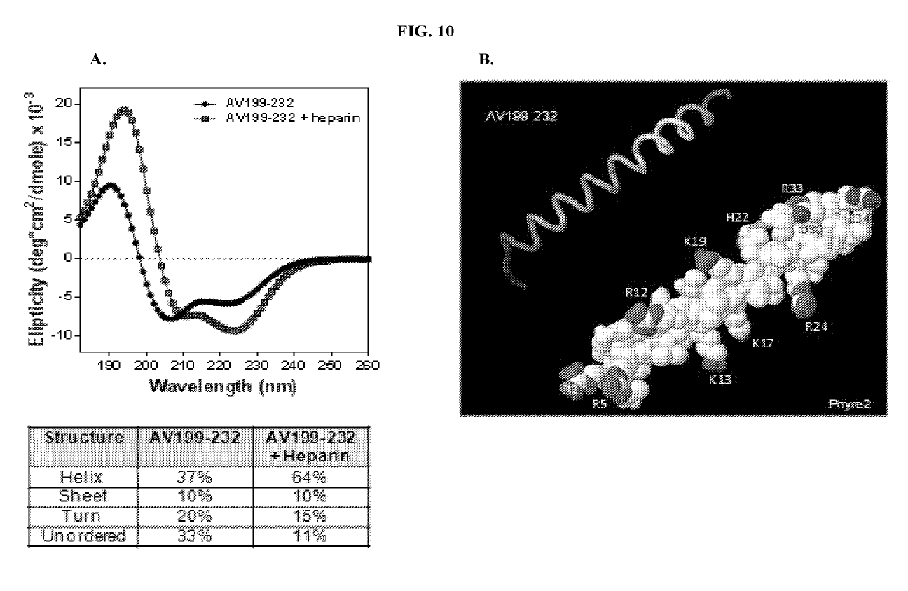

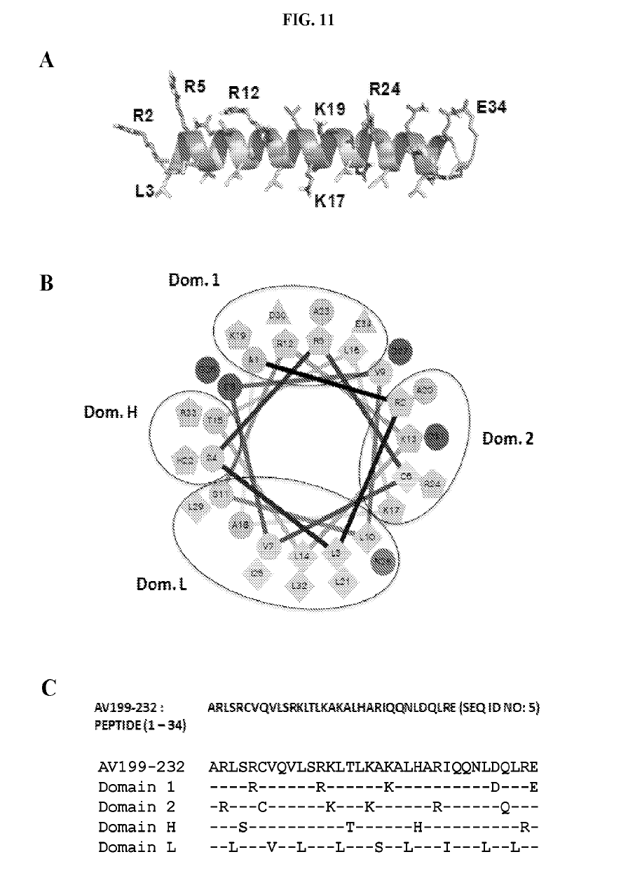

Given that there appeared to be a periodicity in the sequence of AV199-232 consisting of alternating polar and non-polar residues, the possibility that the peptide was folded into a amphipathic α-helix was investigated (FIG. 10). Both AV199-232 and AV199-237 were analyzed by circular dichroism spectroscopy and it was found that AV199-232 contained a-helical structure, the helical nature of which increased on addition of heparin demonstrate heparin binding activity. In silico analysis also predicted a high α-helical content. These data validated the use of a helical wheel presentation of the AV-peptide, which is a common tool used to aid the visualization of the functional domain organization in apo-lipoproteins. The representation of AV199-232 as a helical wheel revealed 3 or 4 discrete domains (FIG. 11). The amphipathic nature of the peptide was evident with polar residues and nonpolar residues (domain-L) organized to opposite sides of the helix. Domain-L is responsible for binding to VLDL. Domain-1, domain-2 and domain-H were also identified on the polar side.

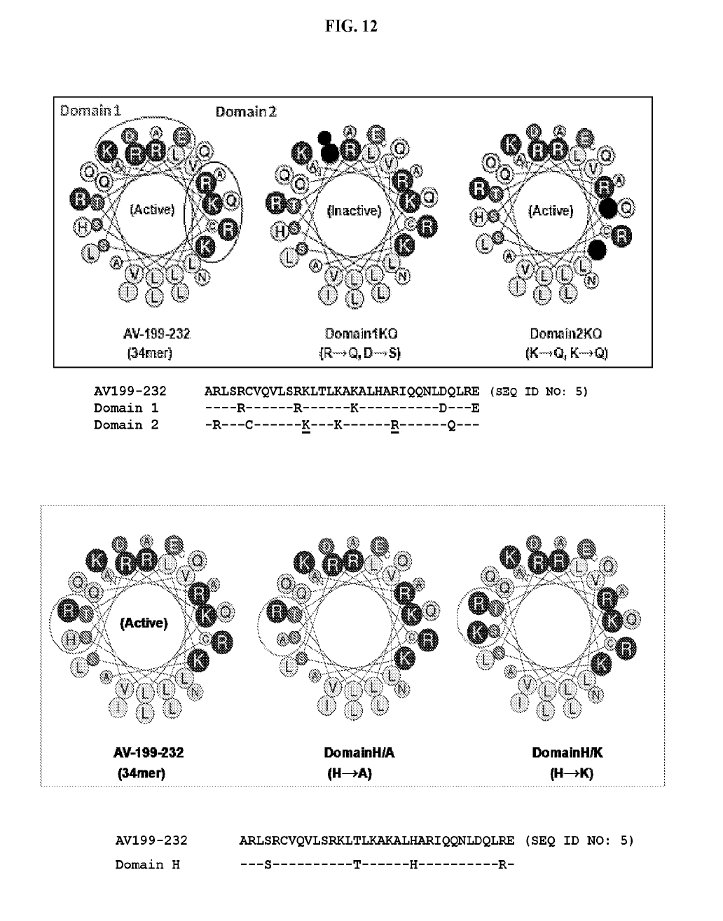

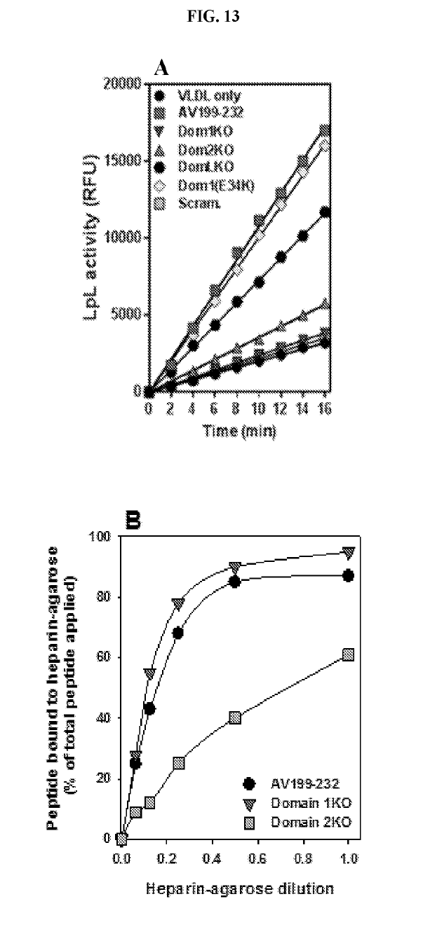

To investigate the potential importance of these domains for lipase activation, a series of peptides were designed in which a limited set of charged residues were replace with a polar uncharged residue compatible with the α-helix structure (FIG. 12). Domain 1 and domain 2 are highly implicated in AV199-232 activity. Substitutions in Dom1 (Dom1KO; R12Q/D30S) and Dom2 (Dom2KO; K13Q/K17Q) resulted in a significant reduction in LpL stimulating activity (FIG. 13). The Dom1(E34K) did not influence AV199-232 activity suggesting that E34 does not play a functional role (FIG. 13A). Substitutions within DomL

(DomLKO; L10Q/L21Q/L29Q) resulted in a moderate reduction in activity (FIG. 13A). Heparin binding activity of the AV199-232, Dom1KO and Dom2KO was compared by applying increasing concentrations of peptide to heparin-Sepharose 4B columns (0.2 mL) (FIG. 13B). Testing these mutant peptides demonstrated that domain-1 was necessary for activation of LpL and domain-2 harbored the heparin binding site and is also highly implicated in activation of LpL (FIG. 13).

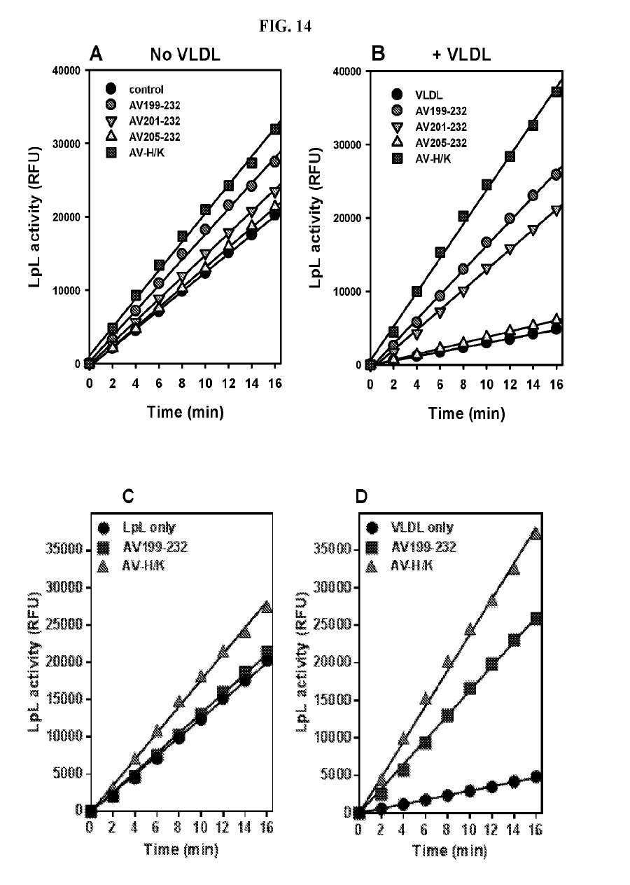

The domain-H was also of interest because unlike the other amino acids it is the only residue that can change from neutral to positive charge at slightly acid pH that can be found close to the cell surface where the lipases are anchored. An AV199-232 peptide with H replaced with a K (AV-H/K) was synthesized and its influence on lipase stimulation assessed (FIG. 14). AV-H/K was able to increase LpL activity in the absence of VLDL by ˜1.6-fold and in the presence of VLDL by ˜8-fold, which corresponds to a 44% enhancement of activity over AV199-232.

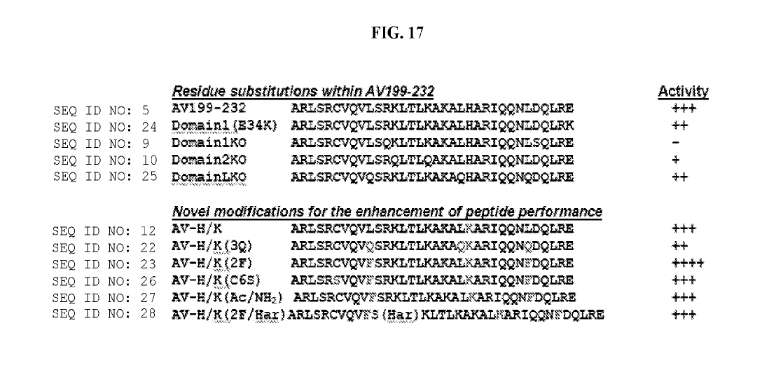

A summary of the main peptides that have been designed and tested for lipase stimulation are listed in FIG. 17. Both CD analysis and in silico predictions strongly support a highly organized a-helical structure for the AV-peptides described herein. This structural information has allowed identification 4-domains not previously recognized in AV199-232, which are consistent with the experiments conducted during development of embodiments described herein, two of which (domain-1 & domain-H) are vital for direct stimulation of lipase activity. In addition, the lipid binding domain-L is also important for activity as demonstrated by the improvement of activity with the inclusion of VLDL. This latter observation has a number of positive implications for the development of AV-peptides as therapeutics. Firstly, TG is normally packaged and circulates in VLDL and other TG-rich lipoproteins and my data suggests that AV-peptides associate with these lipoproteins. Secondly, this affinity for lipoproteins could significantly improve their bioavailability in vivo. Finally, the AV-peptides were able to overcome the inhibition of apoC-III in VLDL and the close proximity to apoC-III maybe critical for this activity. The ability of the AV-peptides to also operate in live cell systems and with different members of the lipase family (e.g., LpL and HL) also indicates that the AV-peptides could have a strong in vivo presence and therapeutic impact on HTG. Finally, the substitution of H with K in domain-H has substantially improved the performance of the AV199-232 peptide. Based on the experiments conducted during development of embodiments of the embodiments described herein, it is contemplated that other modifications within the scope described herein may have similarly improved performance.

### EXAMPLE 4

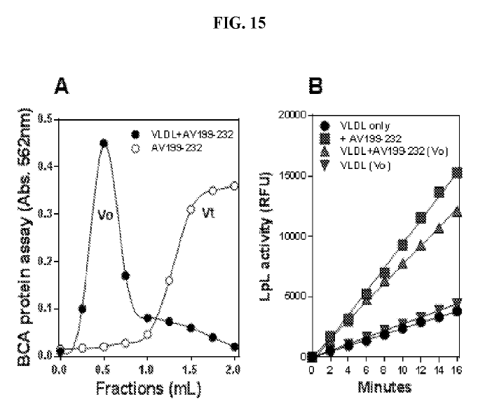

Experiments were conducted during development of embodiments herein to demonstrate that AV199-232 binds VLDL and the peptide-lipoprotein complex stimulates LpL activity. VLDL (100 μg) was incubated with AV199-232 (50 μg) and complexes were isolated from free peptide by gel filtration on a Sepharose CL-4B column (FIG. 15A). The void fraction (Vo) containing the VLDL:AV199-232 complexes were effective at stimulating LpL activity (FIG. 15B).

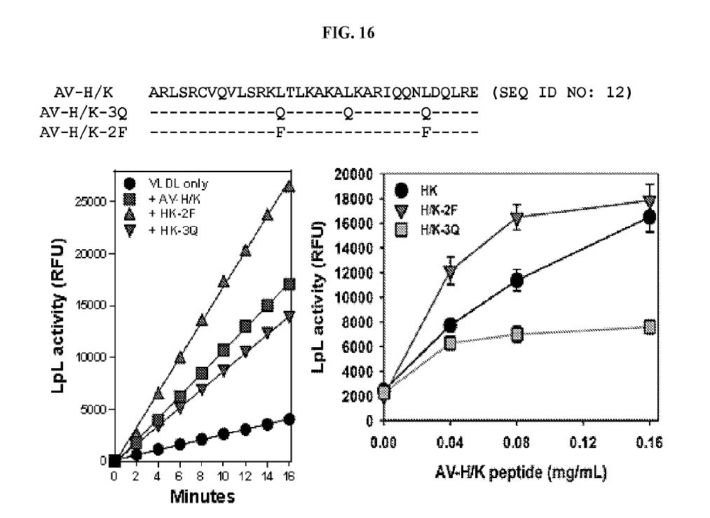

Experiments were conducted during development of embodiments herein to identify the effects of mutating key residues in Domain L of the AV-H/K peptide, and results indicate that substitutions tuning the lipid-binding phase of the AV peptides herein are useful in improving lipase stimulating performance. L10F and L29F substitutions in AV-H/K (SEQ ID NO: 23) result in about 2-fold improvement in lipase stimulating activity, while L10Q, L21Q and L29Q substitutions (SEQ ID NO: 22) reduced lipase stimulating activity (FIG. 16A,B). It is contemplated that L to F and L to Q substitutions in the L Domain increase and decrease binding to VLDL, respectively. Peptides (80 μg/ml) were preincubated with VLDL (20 μg/ml) before the addition of LpL and substrate. Lipase reactions were either continuously monitored for 16 min at room temperature (A), or incubated for 10 min at 37° C. (B).

### EXAMPLE 5

### Alanine Scan of AV199-232

Experiments were conducted during development of embodiments herein to determine the relative importance of the side-chain identity of the individual residues of

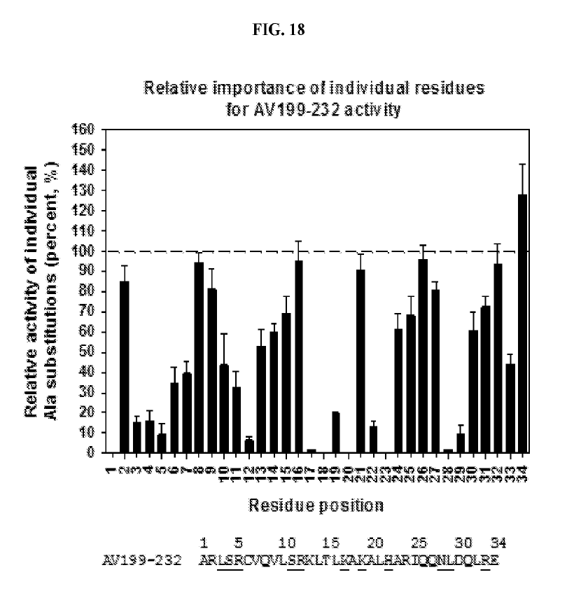

AV199-232. To this end, alanine scanning was conducted on the AV199-232 peptide, individually replacing each residue with an alanine residue (except positions 1, 18, 20, and 23, which are already alanines), and assessing the lipase-stimulating activity of the resulting peptides (FIG. 18). Significant reduction in lipase-stimulating activity identifies a position as important for either direct binding to LpL or as playing an indirect structural role in aiding the correct positioning of LpL stimulating residues in AV199-232. Based on these data, residues are roughly binned into three categories: (1) residues in which replacement with alanine yields >75% lipase stimulating activity of AV199-232, (2) residues in which replacement with alanine yields 25-75% lipase stimulating activity of AV199-232, and (3) residues in which replacement with alanine yields <25% lipase stimulating activity of AV199-232. The rssulkts of the alanine scan indicate that the side chains of residues falling into these three categories are (1) minimally implicated in lipase stimulation, (2) moderately implicated in lipase stimulation, and (3) highly implicated in lipase stimulations. The most highly implicated residues are underlined in the AV199-232 sequence in FIG. 18. These results are consistent with the biochemical studies (Examples 2 and 3), molecular modeling studies (Example 6), and bioinformatics (Example 8) data presented herein.

### EXAMPLE 6

### Molecular Modeling and 3D Docking

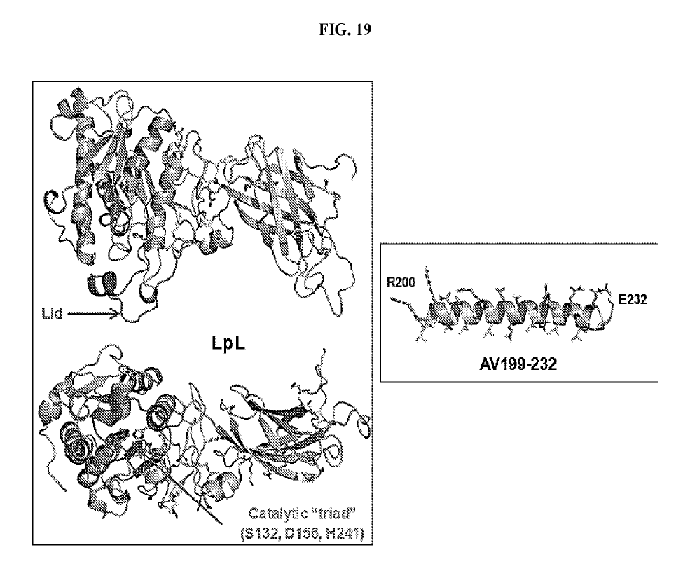

3D structures for LpL (left) and AV199-232 (right) were generated by homology-based modeling using Phyre2 followed by model refinement at GalaxyWEB (FIG. 19). LpL is shown in two poses highlighting different functional regions. The catalytic triad (S132, D156, H241) is located in a “pocket” with an associated 22-residue loop that functions as a lid covering the catalytic pocket and plays a role in substrate recognition (FIG. 19, left/top). AV199-232 model is shown with domain L (hydrophobic lipid-binding domain facing downward) (FIG. 19, right).

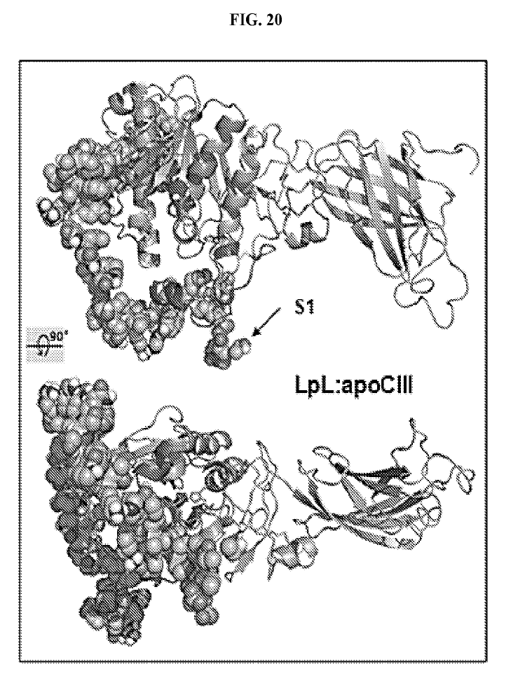

Molecular Docking experiments were performed using ClusPro 2.0 (cluspro.bu.edu/login.php) to evaluate the binding of apoC-III to LpL (FIG. 20). LpL shown in ribbon view and apoC-III are shown with side-chains in sphere format. For apoC-III (PDB ID: 2JQ3) docking is predicted near the lipase catalytic site with N-terminal sequence of apoC-III juxtaposed to the proximal edge of the lid sequence.

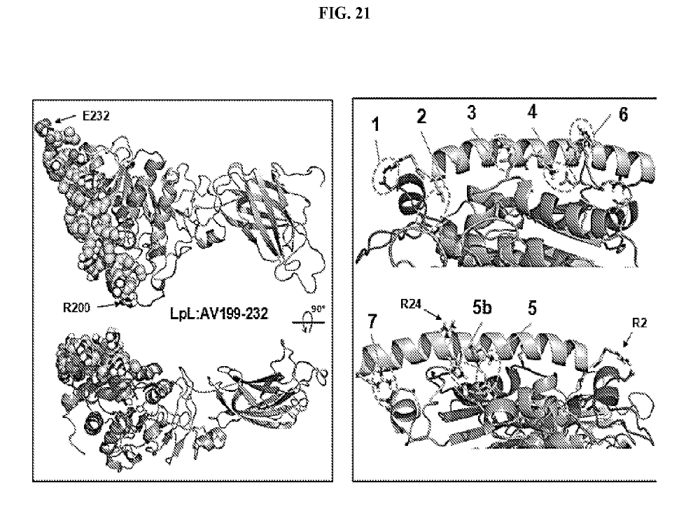

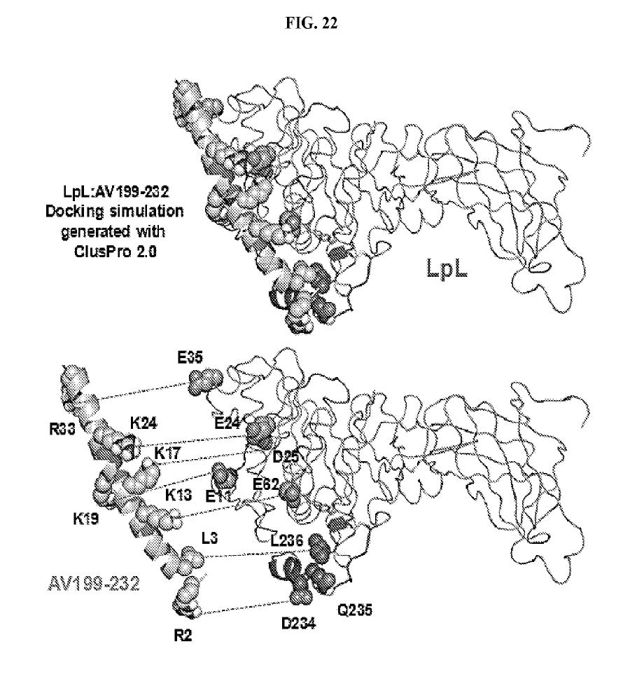

Molecular docking experiments were performed using ClusPro 2.0 (cluspro.bu.edu/login.php) to evaluate the binding of AV199-232 to LpL. The top ranked pose (highest cluster number) is presented (FIGS. 21 and 22) showing the LpL (ribbon (FIG. 21) or backbone (FIG. 22)):AV199-232 (side-chains spheres) complex (left panel). Interactions can be stabilized by side-chains which are ˜4 Å or less apart and forming either salt bridges or H-bonds. Inspection of the LpL:AV199-232 contact points indicates up to 7-residue pairings stabilizing the AV199-232:LpL interactions (right panel); (1) R2-D234 (2.2 Å), (2) L3-L236 (4.8 Å), (3) K13-E62 (1.9 Å), (4) K17-D25 (1.7 Å), (5) K19-E11 (3.1 Å), (5b) H22-E11 (4.2 Å), (6) R24-E24 (2.1Å), and (7) R33-E35 (1.8Å). These pairings are highly consistent with the other experiments presented herein, for example, the alanine scanning results (FIG. 18).

### EXAMPLE 7

### In Vivo Stimulation of LpL Activity

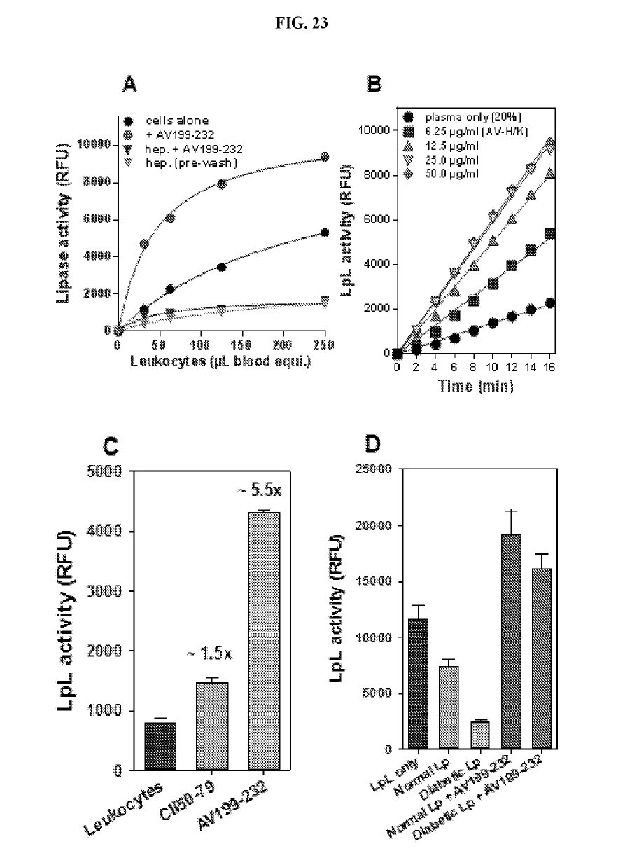

Experiments were conducted during development of embodiments herein to demonstrates that AV-peptides are active in stimulating LpL activity in human blood samples, and thus are expected to be stimulatory in vivo. FIG. 23A demonstrates that LpL in live leukocytes isolated from fresh human blood responds to AV199-232 treatment. The removal of cell surface LpL with heparin reduced activity. AV-H/K also stimulates LpL in the presence of human plasma (FIG. 23B).

Plasma contains lipoproteins with apoC-III and ANGPT4, known inhibitors of LpL activity. However, LpL activity in live leukocytes was stimulated by AV199-232 (FIG. 23C). AV199-232 stimulates LpL activity in the presence of normal and dyslipidemic plasma (FIG. 23D). AV199-232 was incubated with whole plasma from normal and diabetic/dyslipidemic plasma and then the TG-rich lipoproteins (Lp) were isolated by PEG-8000 precipitation and added to LpL assays (30 μg/ml). Experiments demonstrate that AV199-232 associates with and activates LpL in plasma.

### EXAMPLE 8

### AV199-232 Homology Analysis

A homology search was made of the National Center for Biotechnology Information (NCBI) Protein Database to identify orthologues to the AV199-232 peptide sequence, and to identify residues that were either invariant among similar sequences or in which substitutions were permissible. Default parameters on the NCBI BLASTP website were used. The results are organized in Table 1 to identify a set of suitable amino acid substitutions for the residues in the AV199-232 peptide.

TABLE 1

| Residue | | | | |
|---|---|---|---|---|
| number | | | | |
| in | | | | |
| peptide | | | | |
| (and in | | | | |
| full | | | | Proposed |
| length | Amino | Observed | | contact |
| protein) | acid | changes | Domain | site? |
| 1 (199) | A | S, P, G | 1 | |
| 2 (200) | R | K, Q, G | 2 | Yes |
| 3 (201) | L | | L | Yes |
| 4 (202) | S | N, G | H | |
| 5 (203) | R | Q, H, G | 1 | |
| 6 (204) | C | Y, H, D | 2 | |
| 7 (205) | V | M, I | L | |
| 8 (206) | Q | | | |
| 9 (207) | V | T, M, A, I, | | |
| | | L, E, G, S, | | |
| | | D, Q | | |
| 10 (208) | L | | L | |
| 11 (209) | S | | L | |
| 12 (210) | R | Q, S, H, | 1 | |
| | | T, N, K, E, | | |
| | | A, | | |
| 13 (211) | K | | 2 | Yes |
| 14 (212) | L | | L | |
| 15 (213) | T | | H | |
| 16 (214) | L | R, V, F, | 1 | |
| | | Q, W | | |
| 17 (215) | K | Q, E, N, | 2 | Yes |
| | | S, E | | |
| 18 (216) | A | | L | |
| 19 (217) | K | E, Q, R, | 1 | Yes |
| | | G, T, H, | | |
| 20 (218) | A | T, D, E, S | 2 | |
| 21 (219) | L | | L | |
| 22 (220) | H | | H | Yes |
| 23 (221) | A | T, E, S, | 1 | |
| | | Q, H | | |
| 24 (222) | R | G, S, H, K | 2 | Yes |
| 25 (223) | I | V | L | |
| 26 (224) | Q | E, R | | |
| 27 (225) | Q | K, R, H, | | |
| | | T, G | | |
| 28 (226) | N | | L | |
| 29 (227) | L | V | L | |
| 30 (228) | D | E, N | 1 | |
| 31 (229) | Q | R, H, K | 2 | |
| 32 (230) | L | | L | |
| 33 (231) | R | H, L, K, Q | H | Yes |
| 34 (232) | E | D, Q | 1 | |

All publications and patents listed below and/or provided herein are incorporated by reference in their entireties. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

### REFERENCES

The following references, some of which are also cited above by name and/or number, are herein incorporated by reference in their entireties.

1. Do, R., et al., (2013) Common variants associated with plasma triglycerides and risk for coronary artery disease Nat.Genet. 45, 1345-1352

2. Ford, E. S., Li, C., Zhao, G., Pearson, W. S., Mokdad, A. H. (2009) Hypertriglyceridemia and its pharmacologic treatment among US adults. Arch.Intern.Med. 169, 572-578

3. Christian, J. B., Bourgeois, N., Snipes, R., Lowe, K. A. (2011) Prevalence of severe (500 to 2,000 mg/dl) hypertriglyceridemia in United States adults. Am.J.Cardiol. 107, 891-897

4. Pennacchio, L. A., Olivier, M., Hubacek, J. A., Cohen, J. C., Cox, D. R., Fruchart, J. C., Krauss, R. M., Rubin, E. M. (2001) An apolipoprotein influencing triglycerides in humans and mice revealed by comparative sequencing. Science 294, 169-173

5. van der Vliet, H. N., Sammels, M. G., Leegwater, A. C., Levels, J. H., Reitsma, P. H., Boers, W., Chamuleau, R. A. (2001) Apolipoprotein A-V: a novel apolipoprotein associated with an early phase of liver regeneration. J.Biol.Chem. 276, 44512-44520

6. O'Brien, P. J., Alborn, W. E., Sloan, J. H., Ulmer, M., Boodhoo, A., Knierman, M. D., Schultze, A. E., Konrad, R. J. (2005) The novel apolipoprotein A5 is present in human serum, is associated with VLDL, HDL, and chylomicrons, and circulates at very low concentrations compared with other apolipoproteins. Clin.Chem. 51, 351-359

7. Olofsson, S. O. (2005) ApoA-V: the regulation of a regulator of plasma triglycerides. Arterioscler.Thromb.Vasc.Biol. 25, 1097-1099

8. Fruchart-Najib, J., Bauge, E., Niculescu, L. S., Pham, T., Thomas, B., Rommens, C., Majd, Z., Brewer, B., Pennacchio, L. A., Fruchart, J. C. (2004) Mechanism of triglyceride lowering in mice expressing human apolipoprotein A5. Biochem.Biophys.Res.Commun. 319, 397-404

9. Schaap, F. G., Rensen, P. C., Voshol, P. J., Vrins, C., van der Vliet, H. N., Chamuleau, R. A., Havekes, L. M., Groen, A. K., van Dijk, K. W. (2004) ApoAV reduces plasma triglycerides by inhibiting very low density lipoprotein-triglyceride (VLDL-TG) production and stimulating lipoprotein lipase-mediated VLDL-TG hydrolysis. J.Biol.Chem. 279, 27941-27947

10. Sarwar, N., Sandhu, M. S., Ricketts, S. L., Butterworth, A. S., Di, A. E., Boekholdt, S. M., Ouwehand, W., Watkins, H., Samani, N. J., Saleheen, D., Lawlor, D., Reilly, M. P., Hingorani, A. D., Talmud, P. J., Danesh, J. (2010) Triglyceride-mediated pathways and coronary disease: collaborative analysis of 101 studies. Lancet 375, 1634-1639

11. Nilsson, S. K., Heeren, J., Olivecrona, G., Merkel, M. (2011) Apolipoprotein A-V, a potent triglyceride reducer. Atherosclerosis 219, 15-21

12. Pennacchio, L. A., Olivier, M., Hubacek, J. A., Krauss, R. M., Rubin, E. M., Cohen, J. C. (2002) Two independent apolipoprotein A5 haplotypes influence human plasma triglyceride levels. Hum.Mol.Genet. 11, 3031-3038

13. Charriere, S., Bernard, S., Aqallal, M., Merlin, M., Billon, S., Perrot, L., Le, C. E., Sassolas, A., Moulin, P., Marcais, C. (2008) Association of APOAS -1131T>C and S19W gene polymorphisms with both mild hypertriglyceridemia and hyperchylomicronemia in type 2 diabetic patients. Clin.Chim.Acta 394, 99-103

14. Talmud, P. J., Martin, S., Taskinen, M. R., Frick, M. H., Nieminen, M. S., Kesaniemi, Y. A., Pasternack, A., Humphries, S. E., Syvanne, M. (2004) APOAS gene variants, lipoprotein particle distribution, and progression of coronary heart disease: results from the LOCAT study. J.Lipid Res. 45, 750-756

15. Priore, O. C., Pisciotta, L., Li, V. G., Sambataro, M. P., Cantafora, A., Bellocchio, A., Catapano, A., Tarugi, P., Bertolini, S., Calandra, S. (2005) Inherited apolipoprotein A-V deficiency in severe hypertriglyceridemia. Arterioscler.Thromb.Vasc.Biol. 25, 411-417

16. Priore, O. C., Carubbi, F., Schaap, F. G., Bertolini, S., Calandra, S. (2008) Hypertriglyceridaemia and low plasma HDL in a patient with apolipoprotein A-V deficiency due to a novel mutation in the APOAS gene. J.Intern.Med. 263, 450-458

17. Tang, Y., Sun, P., Guo, D., Ferro, A., Ji, Y., Chen, Q., Fan, L. (2006) A genetic variant c.553G >T in the apolipoprotein A5 gene is associated with an increased risk of coronary artery disease and altered triglyceride levels in a Chinese population. Atherosclerosis 185, 433-437

18. Pullinger, C. R., Aouizerat, B. E., Movsesyan, I., Durlach, V., Sijbrands, E. J., Nakajima, K., Poon, A., Dallinga-Thie, G. M., Hattori, H., Green, L. L., Kwok, P. Y., Havel, R. J., Frost, P. H., Malloy, M. J., Kane, J. P. (2008) An apolipoprotein A-V gene SNP is associated with marked hypertriglyceridemia among Asian-American patients. J.Lipid Res. 49, 1846-1854

19. Sharma, V., Witkowski, A., Witkowska, H. E., Dykstra, A., Simonsen, J. B., Nelbach, L., Beckstead, J. A., Pullinger, C. R., Kane, J. P., Malloy, M. J., Watson, G., Forte, T. M., Ryan, R. O. (2014) Aberrant hetero-disulfide bond formation by the hypertriglyceridemia-associated p.Glyl85Cys APOAS variant (rs2075291). Arterioscler.Thromb.Vasc.Biol. 34, 2254-2260

20. Nilsson, S. K., Lookene, A., Beckstead, J. A., Gliemann, J., Ryan, R. O. Olivecrona, G. (2007) Apolipoprotein A-V interaction with members of the low density lipoprotein receptor gene family. Biochemistry 46, 3896-3904

21. Sun, G., Bi, N., Li, G., Zhu, X., Zeng, W., Wu, G., Xue, H., Chen, B. (2006) Identification of lipid binding and lipoprotein lipase activation domains of human apoAV. Chem.Phys.Lipids 143, 22-28

22. Basu, D., Manjur, J., Jin, W. (2011) Determination of lipoprotein lipase activity using a novel fluorescent lipase assay. J.Lipid Res. 52, 826-832

1. A composition comprising a synthetic peptide or polypeptide having less than 100% sequence identity with SEQ ID NO: 1 (full length ApoA-V), encompassing a portion with at least 50% sequence identity SEQ ID NO: 8 (AV199-224), and exhibiting lipase-stimulating activity.

2. The composition of claim 1, wherein the peptide or polypeptide has at least 80% sequence similarity with SEQ ID NO: 8.

3. The composition of claim 1, wherein the peptide or polypeptide has less than 100% sequence identity, but more than 50% sequence identity with SEQ ID NO: 3 (AV199-237).

4. The composition of claim 3, wherein the peptide or polypeptide has at least 80% sequence similarity with SEQ ID NO: 3.

5. The composition of claim 1, comprising a peptide with less than 100% sequence identity, but more than 50% sequence identity with SEQ ID NO: 5 (AV199-232).

6. The composition of claim 5, wherein the peptide has at least 80% sequence similarity with SEQ ID NO: 5.

7. The composition of claim 5, wherein the peptide has less than 100% sequence identity, but more than 50% sequence identity with SEQ ID NO: 10.

8. The composition of claim 7, wherein the peptide has at least 80% sequence similarity with SEQ ID NO: 10.

9. The composition of claim 5, wherein the peptide has less than 100% sequence identity, but more than 50% sequence identity with SEQ ID NO: 11.

10. The composition of claim 9, wherein the peptide has at least 80% sequence similarity with SEQ ID NO: 11.

11. The composition of claim 5, wherein the peptide has less than 100% sequence identity, but more than 50% sequence identity with SEQ ID NO: 12.

12. The composition of claim 11, wherein the peptide has at least 80% sequence similarity with SEQ ID NO: 12.

13. The composition of one of claims 1-12, wherein the peptide or polypeptide exhibits enhanced lipase-stimulating activity over SEQ ID NO: 1 (full length ApoA-V).

14. A pharmaceutical preparation comprising: (a) a peptide or polypeptide of one of claims 1-13; and (b) a physiologically acceptable buffer or carrier.

15. A fusion peptide or polypeptide comprising a peptide or polypeptide of one of claims 1-13 and a functional peptide or polypeptide segment.

16. The fusion peptide or polypeptide of claim 15, wherein the functional peptide or polypeptide segment comprises a signaling moiety, therapeutic moiety, localization moiety, detectable moiety, or isolation/purification moiety.

17. A polynucleotide encoding peptide or polypeptide of one of claims 1-13.

18. A nucleic acid vector comprising the polynucleotide of claim 17.

19. The nucleic acid vector of claim 18, further comprising a promoter and/or one or more expression elements.

20. A method of treating hypertriglyceridemia or a related condition or disease comprising administering a peptide or polypeptide of one of claims 1-13 to a subject suffering from hypertriglyceridemia or said related condition or disease.

21. A method of preventing hypertriglyceridemia or a related condition or disease comprising administering a peptide or polypeptide of one of claims 1-13 to a subject at risk of hypertriglyceridemia or said related condition or disease.

22. A method of treating or preventing atherosclerosis and/or cardiovascular disease in a subject comprising administering a peptide or polypeptide of one of claims 1-13 to a subject.

23. The method of claim 22, comprising co-administering: (a) said peptide or polypeptide, and (b) a therapy and/or therapeutic for the treatment and/or prevention of atherosclerosis and/or cardiovascular disease.

24. The method of claim 23, wherein (a) and (b) are administered simultaneously and/or in a single pharmaceutical preparation.

25. The method of claim 23, wherein (a) and (b) are administered concurrently and/or in separate pharmaceutical preparations.

26. A method of reducing triglyceride concentration in a sample comprising:
(a) administering a peptide or polypeptide of one of claims 1-13 to a sample comprising (i) triglycerides, and (ii) triglyceride-hydrolyzing lipases; or
(b) administering: (i) a peptide or polypeptide of one of claims 1-13, and (ii) triglyceride-hydrolyzing lipases to a sample comprising triglycerides.

27. The method of claim 26, the triglyceride-hydrolyzing lipases selected from the group consisting of lipoprotein lipase (LpL), endothelial lipase (EL) and hepatic lipase (HL).


SEQUENCE LISTING (29 sequences)

SEQ ID NO: 1 - protein, 343 aa, Homo sapiens
RKGFWDYFSQTSGDKGRVEQIHQQKMAREPATLKDSLEQDLNNMNKFLEKLRPLSGSEAPRLPQDPVGMRRQLQEELEEVKARLQPYMAEAHELVGWNLEGLRQQLKPYTMDLMEQVALRVQELQEQLRVVGEDTKAQLLGGVDEAWALLQGLQSRVVHHTGRFKELFHPYAESLVSGIGRHVQELHRSVAPHAPASPARLSRCVQVLSRKLTLKAKALHARIQQNLDQLREELSRAFAGTGTEEGAGPDPQMLSEEVRQRLQAFRQDTYLQIAAFTRAIDQETEEVQQQLAPPPPGHSAFAPEFQQTDSGKVLSKLQARLDDLWEDITHSLHDQGHSHLGDP

SEQ ID NO: 2 - protein, 60 aa, Homo sapiens
PHAPASPARLSRCVQVLSRKLTLKAKALHARIQQNLDQLREELSRAFAGTGTEEGAGPDP

SEQ ID NO: 3 - protein, 39 aa, Homo sapiens
ARLSRCVQVLSRKLTLKAKALHARIQQNLDQLREELSRA

SEQ ID NO: 4 - protein, 29 aa, Homo sapiens
SRKLTLKAKALHARIQQNLDQLREELSRA

SEQ ID NO: 5 - protein, 34 aa, Homo sapiens
ARLSRCVQVLSRKLTLKAKALHARIQQNLDQLRE

SEQ ID NO: 6 - protein, 32 aa, Homo sapiens
LSRCVQVLSRKLTLKAKALHARIQQNLDQLRE

SEQ ID NO: 7 - protein, 28 aa, Homo sapiens
VQVLSRKLTLKAKALHARIQQNLDQLRE

SEQ ID NO: 8 - protein, 26 aa, Homo sapiens
ARLSRCVQVLSRKLTLKAKALHARIQ

SEQ ID NO: 9 - protein, 34 aa, Artificial sequence
  Mutated wild-type peptide
ARLSRCVQVLSQKLTLKAKALHARIQQNLSQLRE

SEQ ID NO: 10 - protein, 34 aa, Artificial sequence
  Mutated wild-type peptide
ARLSRCVQVLSRQLTLQAKALHARIQQNLDQLRE

SEQ ID NO: 11 - protein, 34 aa, Artificial sequence
  Mutated wild-type peptide
ARLSRCVQVLSRKLTLKAKALQARIQQNLDQLRE

SEQ ID NO: 12 - protein, 34 aa, Artificial sequence
  Mutated wild-type peptide
ARLSRCVQVLSRKLTLKAKALKARIQQNLDQLRE

SEQ ID NO: 13 - protein, 34 aa, Artificial sequence
  Mutated wild-type peptide
ARLSRAVQVLSRKLTLKAKALKARIQQNLDQLRE

SEQ ID NO: 14 - protein, 34 aa, Artificial sequence
  Mutated wild-type peptide
ARLSRAVQVLSRRLTLQAKALHAKIQENLDDLRE

SEQ ID NO: 15 - protein, 34 aa, Artificial sequence
  Mutated wild-type peptide
  MISC_FEATURE (13)..(13): Xaa = any amino acid
  MISC_FEATURE (17)..(17): Xaa = any amino acid
  MISC_FEATURE (22)..(22): Xaa = any amino acid
ARLSRCVQVLSRXLTLXAKALXARIQQNLDQLRE

SEQ ID NO: 16 - protein, 34 aa, Artificial sequence; identical to SEQ ID NO: 15
  Mutated wild-type peptide
  MISC_FEATURE (13)..(13): Xaa = any amino acid
  MISC_FEATURE (17)..(17): Xaa = any amino acid
  MISC_FEATURE (22)..(22): Xaa = any amino acid

SEQ ID NO: 17 - protein, 34 aa, Artificial sequence; identical to SEQ ID NO: 15
  Mutated wild-type peptide
  MISC_FEATURE (13)..(13): Xaa = K or Q
  MISC_FEATURE (17)..(17): Xaa = K or Q
  MISC_FEATURE (22)..(22): Xaa = H, K or Q

SEQ ID NO: 18 - protein, 93 aa, Homo sapiens
PASPARLSRCVQVLSRKLTLKAKALHARIQQNLDQLREELSRAFAGTGTEEGAGPDPQMLSEEVRQRLQAFRQTYLQIAAFTRAIDQETEEVQ

SEQ ID NO: 19 - protein, 39 aa, Homo sapiens
DPQMLSEEVRQRLQAFRQDTYLQIAAFTRAIDQETEEVQ

SEQ ID NO: 20 - protein, 32 aa, Homo sapiens
ARLSRCVQVLSRKLTLKAKALHARIQQNLDQL

SEQ ID NO: 21 - protein, 34 aa, Artificial sequence
  Mutated wild-type peptide
VQHACKRLDKSRALSRLLLTLRQELRKQANVQAI

SEQ ID NO: 22 - protein, 34 aa, Artificial sequence
  Mutated wild-type peptide
ARLSRCVQVQSRKLTQKAKALKARIQQNQDQLRE

SEQ ID NO: 23 - protein, 34 aa, Artificial sequence
  Mutated wild-type peptide
ARLSRCVQVFSRKLTLKAKALKARIQQNFDQLRE

SEQ ID NO: 24 - protein, 34 aa, Artificial sequence
  Mutated wild-type peptide
ARLSRCVQVLSRKLTLKAKALHARIQQNLDQLRK

SEQ ID NO: 25 - protein, 34 aa, Artificial sequence
  Mutated wild-type peptide
ARLSRCVQVQSRKLTLKAKAQHARIQQNQDQLRE

SEQ ID NO: 26 - protein, 34 aa, Artificial sequence
  Mutated wild-type peptide
ARLSRSVQVFSRKLTLKAKALKARIQQNFDQLRE

SEQ ID NO: 27 - protein, 34 aa, Artificial sequence; identical to SEQ ID NO: 23
  Mutated wild-type peptide

SEQ ID NO: 28 - protein, 34 aa, Artificial sequence
  Mutated wild-type peptide
  MISC_FEATURE (12)..(12): Xaa = homoArginine
ARLSRCVQVFSXKLTLKAKALKARIQQNFDQLRE

SEQ ID NO: 29 - protein, 34 aa, Artificial sequence
  Mutated wild-type peptide
  MISC_FEATURE (3)..(3): Xaa = L, F, Y, or W
  MISC_FEATURE (4)..(4): Xaa = S, T, N, or G
  MISC_FEATURE (5)..(5): Xaa = R, K, homoLysine, or homoArginine
  MISC_FEATURE (6)..(6): Xaa = C, S, T, N, Q, Y, or H
  MISC_FEATURE (7)..(7): Xaa = V, F, Y, or W
  MISC_FEATURE (10)..(10): Xaa = L, F, Y, or W
  MISC_FEATURE (12)..(12): Xaa = R, K, hLys, or hArg
  MISC_FEATURE (13)..(13): Xaa = K, R, hArg, hLys, or Orn
  MISC_FEATURE (14)..(14): Xaa = L, F, Y, or W
  MISC_FEATURE (17)..(17): Xaa = K, R, hArg, hLys, or Orn
  MISC_FEATURE (18)..(18): Xaa = A, F, Y, or W
  MISC_FEATURE (19)..(19): Xaa = R, K, hLys, or hArg
  MISC_FEATURE (21)..(21): Xaa = L, F, Y, or W
  MISC_FEATURE (22)..(22): Xaa = H, K, hLys, R, hArg, or Orn
  MISC_FEATURE (25)..(25): Xaa = I, F, Y, or W
  MISC_FEATURE (29)..(29): Xaa = L, F, Y, or W
  MISC_FEATURE (32)..(32): Xaa = L, F, Y, or W
ARXXXXXQVXSXXXTLXXXAXXARXQQNXDQXRE